(19) United States
(12) Patent Application Publication
(10) Pub. No.: US 2020/0101085 A1
(43) Pub. Date: Apr. 2, 2020

(54) TRANSPORE DELIVERY OF DRUGS AND USES THEREOF

(71) Applicant: Joel STUDIN, Roslyn, NY (US)

(72) Inventor: Joel STUDIN, Roslyn, NY (US)

(21) Appl. No.: 16/586,690

(22) Filed: Sep. 27, 2019

Related U.S. Application Data
(60) Provisional application No. 62/738,842, filed on Sep. 28, 2018.

(51) Int. Cl. A61K 31/568; A61K 47/26; A61K 47/10; A61K 9/00; A61K 9/70; A61K 31/485; A61K 31/455; A61K 31/565; A61K 31/573; A61K 31/137; A61P 25/34

(52) U.S. Cl. CPC A61K 31/568; A61K 47/26; A61K 47/10; A61K 9/0014; A61K 9/7015; A61P 25/34; A61K 31/455; A61K 31/565; A61K 31/573; A61K 31/137; A61K 31/485

(57) ABSTRACT

This invention is related to novel topical compositions and methods thereof for delivery of drugs to a subject, more specifically to deliver sex hormones, opioids, antidiabetics, smoking cessation agents, and psychoactive drugs to a patient via skin pores.

This application claims priority benefit to U.S. Provisional Application No. 62/738,842, filed Sep. 28, 2018, the content of which is hereby incorporated by reference in its entirety.

### FIELD OF THE INVENTION

The present invention relates to methods for transpore delivery of novel topical liquid compositions to a subject in need thereof. More specifically, the topical compositions comprise drugs or proteins such as sex hormones, opioids, anti-diabetics, smoking cessation agents, or psychoactive drugs.

### BACKGROUND

Topical delivery of drugs or proteins such as sex hormones, opioids, anti-diabetics, smoking cessation agents, or psychoactive drugs is often desired but difficult to achieve.

Low levels of sex hormones can lead to many disturbances in the body. For example, a low level of testosterone may cause impaired sexual function and/or libido, metabolic syndrome which may be a combination of abdominal obesity, high blood pressure, insulin resistance, lipid disorders; high risk of cardiovascular diseases; reduced bone mass/mineral density and muscle weakness and or degeneration affecting the musculoskeletal system. Other effects of low testosterone levels include negative changes in body composition, depression and other psychological disorders.

Similarly, low levels of estrogen may cause decreased estrogenization of the vulvo-vaginal tissue and cause vaginal dryness, vaginal odor, vaginal or vulva-irritation or itching, dysuria (pain, burning, or stinging when urinating), dyspareunia (vaginal pain associated with sexual activity), or vaginal bleeding associated with sexual activity. Other symptoms of low levels of estrogen include soreness with urinary frequency and urgency, urinary discomfort and incontinence”′. Estrogen replacement therapy has proven to be successful in relieving these symptoms.

Opioids remain key agents for the treatment of a wide variety of acute and chronic pain. The World Health Organization has recommended morphine as the analgesic of choice for the treatment of severe cancer pain. Additionally, morphine and related opioids are widely used to alleviate moderate to severe pain after surgery or trauma, or associated with medical illness. Patients with apparently similar pain states can have large differences in opioid dosing requirements. Factors that contribute to this variability include psychosocial status, type of pain (nociceptive, inflammatory, neuropathic or mixed) and its severity, concurrent medications, gender and other genetic aspects, and whether patients are opioid-naïve or tolerant.

Cigarette smoking may produce many undesired effects. The combustion process of tobacco is complex with about 4,000 compounds being generated during combustion. Among the compounds being generated are those which produce highly undesirable effects such as carbon monoxide, carbon dioxide, nitrogen oxides, ammonia, and many other substances. In addition, many substances are left in the lungs as “tar.” The variety of substances generated by burning tobacco include many which are believed to have serious long term health effects. Because of these and other undesirable side effects, attempts have been made to provide acceptable nicotine substitutes to tobacco cigarettes, such as electronic cigarettes, patches, creams, chewing gums and lozenges.

Diabetes is a disease characterized by failure of insulin feedback and secretion in the beta cells of the pancreatic islets of Langerhans and is one of the most common endocrine diseases across all age groups and populations. The most obvious metabolic effect in diabetes is chronic, erratic elevation of the blood glucose level which is associated with progressive damage to blood vessels. This may lead to heart attack, stroke, blindness, peripheral nerve dysfunction, and kidney failure. Insulin is the mainstay for treatment of virtually all Type-I and many Type-II diabetic patients. When necessary, insulin may be administered intravenously or intramuscularly.

Transdermal drug delivery can be attempted through various dosage forms, for example, patches, creams and ointments. Each dosage form has its respective limitations. Patches are difficult to apply on curved surfaces, cumbersome, and uncomfortable. They also cause pain when peeled off and have poor aesthetic appeal. Dermal patches also exhibit reliability problems, not sticking predictably in different climates and degrees of skin oiliness. This limits the efficacy of transdermal drug delivery via patches. It is well-established that amplification of transdermal bioavailability by occlusion alone is inadequate to treat many maladies.

Semisolid preparations, like creams and ointments, overcome some of these drawbacks but have other limitations. Creams and ointments do not allow persistent contact with the skin, can be easily wiped off, and need to be applied repeatedly. They also leave a sticky and greasy feel after application, which may lead to poor patient compliance. Moreover, when creams and ointments are applied to skin, there is a risk of the drug transferring to another person. This is especially true for cream and ointment products containing testosterone or estrogen.

Additionally, as with transdermal patches, it is well-established that bioavailability of the active drug is often inadequate for treatment when delivered by creams and ointments. In these situations, only injections have been effective. Injections, however, are painful, expensive, and poorly tolerated by patients, especially when there is a need for repeat injections over time.

Therefore, there is a need for an improved method of transpore drug delivery of drugs, bypassing the skin surface without an injection, which permits painless, comfortable and invisible application and thereby improves drug delivery and patient compliance.

### BRIEF SUMMARY OF THE INVENTION

According to various aspects and embodiments of this disclosure, the present invention is directed to topical pharmaceutical compositions for transpore delivery and methods of use.

In some embodiments, the active ingredient is delivered through skin pores, bypassing the stratum corneum of the skin, and into the systemic circulation of a subject.

In some embodiments, the present disclosure provides a topical pharmaceutical composition including, but not limited to, hormones such as cortisone, adrenaline, or sex hormones, opioids, anti-diabetics, smoking cessation agents, and/or psychoactive drugs. In other aspect, the present disclosure provides a method of systemically delivering active agents to a subject in need thereof via transpore delivery.

The present disclosure provides a method of transpore delivery of testosterone to a subject in need of testosterone or replacement therapy, the method comprising applying a liquid composition comprising about 0.5% to about 4% by weight of testosterone to the skin of the subject, wherein the liquid testosterone composition, when administered to the subject, achieves one or more of the following: a) has a thickness of about 0.1 μm to about 10 μm in solid or semi-solid form; b) forms a solid or semi-solid film; and c) provides a mean Tₘₐₓ of testosterone from about 1 hour to about 10 hours; wherein the liquid testosterone composition seeps into skin pores and creates a biomechanical integration with the interior of said skin pores in solid form.

In some embodiments, after the liquid testosterone composition is applied to the skin, the film has a thickness of about 1 μm to about 5 μm in solid form. In one embodiment, the film is an occlusive film.

In some embodiments, the area of skin application is about 1 cm² to about 500 cm².

In some embodiments, administration results in at least 10% of the applied testosterone entering the systemic circulation of the subject after about 10 to about 24 hours of contact on the skin.

In some embodiments, when applied to the skin of the subject, the testosterone composition provides a maximum serum testosterone concentration (Cₘₐₓ) following administration of from about 300 ng/dL to about 2500 ng/dL of testosterone. In some embodiments, the maximum serum testosterone concentration (Cₘₐₓ) following administration is from about 400 ng/dL to about 2000 ng/dL. In some embodiments, the maximum serum testosterone concentration (Cₘₐₓ) following administration is from about 500 ng/dL to about 800 ng/dL of testosterone. In other embodiments, the composition provides a mean plasma concentration of testosterone following administration from about 300 ng/dL to about 1100 ng/dL of testosterone.

In some embodiments, the subject is a mammal. In a preferred embodiment, the subject is a human. In another embodiment, the human is a human male. In a further embodiment, the human male is an adult. In another embodiment, the human male is at the age of above 50. In some embodiments, the human suffers from one or more of the following conditions: congenital or acquired primary hypogonadism, hypogonadotropic hypogonadism, cryptorchidism, bilateral torsion, orchitis, vanishing testis syndrome, orchidectomy, Klinefelter's syndrome, post castration, eunuchoidism, hypopituitarism, endocrine impotence, infertility due to spermatogenic disorders, impotence, or male sexual dysfunction. In yet another embodiment, the human suffers from idiopathic gonadotropin, LHRH deficiency, or pituitary hypothalamic injury from tumors, trauma, or radiation. In one embodiment, the human male adult has morning testosterone levels less than 300 ng/dL.

In some embodiments, the liquid testosterone composition is applied to the skin. In some embodiments, the site for application to the skin includes, but is not limited to, an anxilla (underarm), the shoulder, or upper arm.

In some embodiments, the testosterone composition is brushed on to the skin once or twice a day in an amount of from about 0.5 mL to about 5.0 mL. In some embodiments, the testosterone composition is brushed on to the skin one to four times a day for a period of time from 1 day to 365 days. In a preferred embodiment, the testosterone composition is brushed on to the skin once a day for about 120 days. In some embodiments, about 0.1 gram to about 10 grams of the testosterone composition are brushed to the skin each time.

In some embodiments, the ratio of peak concentration and trough concentration of testosterone in the serum of the subject after application is less than 10.

The present disclosure also provides a liquid composition for topical administration comprising about 0.5% to about 4% by weight of testosterone, wherein the composition, when administered to a subject, achieves one or more of the following: a) has a thickness of about 0.1 μm to about 10 μm in solid or semi-solid form; b) forms a solid or semi-solid film; and c) provides a mean Tₘₐₓ of testosterone from about 1 hour to about 10 hours. The testosterone composition seeps into skin pores in liquid form and creates a biomechanical integration with the interior of said skin pores in solid form. In some embodiments, the composition further comprises pyroxilin, ether, and alcohol.

In some embodiments, the testosterone composition, when applied to the skin of the subject, provides a maximum serum testosterone concentration (Cₘₐₓ) following administration from about 300 ng/dL to about 1100 ng/dL of testosterone, about 400 ng/dL to about 900 ng/dL of testosterone, or about 500 ng/dL to about 800 ng/dL of testosterone.

The present disclosure also provides a method of transpore delivery of estrogen to a subject in need of estrogen or replacement therapy, the method comprising applying a liquid composition comprising about 0.5% to about 4% by weight of estrogen to the skin of the subject, wherein the estrogen liquid composition, when administered to the subject, achieves one or more of the following: a) has a thickness of about 0.1 to about 10 μm in solid form; b) forms a solid or semi-solid film; and c) provides a mean Tₘₐₓ of estrogen from about 10 to about 24 hours;, wherein the liquid estrogen composition seeps into skin pores and creates a biomechanical integration with the interior of said skin pores in solid form.

In some embodiments, the area of skin application is about 1 cm² to about 500 cm ².

In some embodiments, administration results in at least 10% of the applied estrogen entering the systemic circulation of the subject after about 10 to about 24 hours of contact on the skin.

In some embodiments, after the liquid estrogen composition is applied to the skin, the film formed has a thickness of about 1 μm to about 5 μm. In another embodiment, the film is an occlusive film.

In some embodiments, the liquid estrogen composition provides a maximum serum estrogen concentration (Cₘₐₓ) following administration from about 20 pg/mL to about 350 pg/mL of estrogen. In some embodiments, the maximum serum estrogen concentration (Cₘₐₓ) following administration is from about 30 pg/mL to about 300 pg/mL of estrogen. In some embodiments, the maximum serum estrogen concentration (Cₘₐₓ) following administration is from about 40 pg/mL to about 200 pg/mL of estrogen. In some embodiments, the estrogen composition provides a mean plasma concentration of estrogen following administration from about 30 pg/mL to about 150 pg/mL of estrogen.

In some embodiments, the subject is a mammal. In a preferred embodiment, the subject is a human. In another embodiment, the human is a human female. In a further embodiment, the human female is an adult. In some embodiments, the human female is at the age of above 50. In a preferred embodiment the human is a postmenopausal woman.

In other embodiments, the human female suffers from one or more of the following conditions: urogenital atrophy, atrophy of the breasts, cardiovascular disease, changes in hair distribution, thickness of hair, changes in skin condition, or osteoporosis.

In some embodiments, the estrogen composition is applied to the skin. In some embodiments, the site for application to the skin includes, but is not limited to, a forearm, a buttock, or an abdomen.

In some embodiments, the estrogen composition is brushed on to the skin once or twice a day in an amount of from about 0.5 mL to about 5.0 mL. In some embodiments, the estrogen composition is brushed on to the skin one to four times a day for a period of time from 1 day to 365 days. In a preferred embodiment, the estrogen composition is brushed on to the skin once a day for about 84 days. In some embodiments, about 0.1 gram to about 10 grams of the estrogen composition is brushed to the skin each time.

In some embodiments, the ratio of peak concentration and trough concentration of estrogen in the serum of the subject after application is less than about 10.

The present disclosure also provides a liquid composition for topical administration comprising about 0.1% to about 20% by weight of estrogen, wherein the composition, when administered to a subject, achieves one or more of the following: a) has a thickness of about 0.1 μm to about 10 μm in solid or semi-solid form; b) forms a solid or semi-solid film; and c) provides a mean Tₘₐₓ of estrogen from about 10 hours to about 24 hours. In some embodiments, the liquid estrogen composition further comprises pyroxilin, ether, and alcohol. The liquid estrogen composition seeps into skin pores in liquid form and creates a biomechanical integration with the interior of said skin pores in solid form.

In some embodiments, the liquid estrogen composition when administered to the subject, provides a maximum serum estrogen concentration (Cₘₐₓ) following administration from about 20 pg/mL to about 350 pg/mL of estrogen, about 30 pg/mL to about 300 pg/mL of estrogen, or about 40 pg/mL to about 200 pg/mL of estrogen.

The present disclosure also provides a method of transpore delivery of an opioid to treat pain in a subject. The method comprises applying a liquid composition comprising about 0.1% to about 20% by weight of opioid to the skin of the subject, the opioid composition, when administered to the subject, achieves one or more of the following: a) has a thickness of about 0.1 μm to about 10 μm in solid form; b) forms a solid or semi-solid film; and c) provides a mean Tₘₐₓ of opioid from about 1 day to about 7 days; wherein the opioid composition seeps into skin pores in liquid form and creates a biomechanical integration with the interior of said skin pores in solid form.

In some embodiments, the area of skin application is about 1 cm² to about 500 cm ².

In some embodiments, administration results in at least 10% of the applied opioid entering the systemic circulation of the subject after about 10 to about 24 hours of contact on the skin.

In some embodiments, the film has a thickness of about 1 to about 5 μm. In one embodiment, the film is an occlusive film.

In some embodiments, when applied to the skin of the subject, the liquid opioid composition provides a maximum serum opioid concentration (Cₘₐₓ) following administration from about 100 pg/mL to about 1000 pg/mL of opioid. In some embodiments, the maximum serum opioid concentration (Cₘₐₓ) following administration is from about 200 pg/mL to about 700 pg/mL of opioid. In some embodiments, the maximum serum opioid concentration (Cₘₐₓ) following administration is from about 300 pg/mL to about 400 pg/mL of opioid. In some embodiments, the opioid composition provides a mean plasma concentration of opioid following administration from about 50 pg/mL to about 1000 pg/mL of opioid.

In some embodiments, when the liquid opioid composition is administrated to a subject, the subject is a mammal. In a preferred embodiment, the subject is a human. In some embodiments, the human is a patient suffering acute pain. In some embodiments, the human is a patient suffering chronic pain. In some embodiments, the human is a patient suffering from a mixed pain state. In a preferred embodiment, the human is a patient whose pain is severe enough to require daily, around-the-clock, long-term opioid treatment and for which alternative treatment options are inadequate.

In some embodiments, the liquid opioid composition is applied to the skin. In some embodiments, the site for application to the skin includes, but is not limited to, an upper arm, an upper chest, or an upper back.

In some embodiments, the liquid opioid composition is brushed on to the skin once or twice a day in an amount of from about 0.5 mL to about 5.0 mL. In some embodiments, the liquid opioid composition is brushed on to the skin once, twice, three, or four times a day for a period of 1 to 365 days. In a preferred embodiment, the liquid opioid composition is brushed on to the skin once a day for about 84 days. In some embodiments, about 0.1 gram to about 10 grams of the opioid composition are brushed on the skin each time.

In some embodiments, the ratio of peak concentration and trough concentration of opioid in the serum of the subject after application is less than about 10.

The present disclosure also provides a liquid opioid composition comprising about 0.1% to about 20% by weight of opioid, the composition, when administered to a subject, achieves one or more of the following: a) has a thickness of about 0.1 μm to about 10 μm in solid or semi-solid form; b) forms a solid or semi-solid film; and c) provides a mean Tₘₐₓ of opioid from about 1 day to about 7 days. In some embodiments, the liquid opioid composition further comprises pyroxilin, ether, and alcohol. The liquid opioid composition seeps into skin pores in liquid form and creates a biomechanical integration with the interior of said skin pores in solid form.

In some embodiments, the liquid opioid composition, when administered to the subject, provides a maximum serum opioid concentration (Cₘₐₓ) following administration from about 100 pg/mL to about 1000 pg/mL of opioid, about 200 pg/mL to about 700 pg/mL of opioid, or about 300 pg/mL to about 400 pg/mL of opioid.

The present disclosure also provides a method of transpore delivery of nicotine to a subject, the method comprising applying a liquid composition comprising about 0.1% to about 20% by weight of nicotine to the skin of a human; the nicotine composition, when administered to the subject, achieves one or more of the following: a) has a thickness of about 0.1 μm to about 10 μm in solid form; b) forms a solid or semi-solid film; and c) provides a mean Tₘₐₓ of nicotine from about 1 hour to about 24 hours; wherein the nicotine composition seeps into skin pores in liquid form and creates a biomechanical integration with the interior of said skin pores in solid form.

In some embodiments, the area of skin application is about 1 cm² to about 500 cm ².

In some embodiments, administration results in at least 10% of the applied nicotine entering the systemic circulation of the subject after about 10 to about 24 hours of contact on the skin.

In some embodiments, the film has a thickness of about 1 μm to about 5 μm. In one embodiment, the film is an occlusive film.

In some embodiments, the nicotine composition provides a maximum serum nicotine concentration (Cₘₐₓ) following administration from about 5.0 ng/mL to about 50.0 ng/mL of nicotine. In some embodiments, the maximum serum nicotine concentration (Cₘₐₓ) following administration is from about 10.0 ng/mL to about 20 ng/mL of nicotine. In some embodiments, the maximum serum nicotine concentration (Cₘₐₓ) following administration is from about 15 ng/mL to about 20 ng/mL of nicotine. In some embodiments, the nicotine composition provides a mean plasma concentration of nicotine following administration from about 2.5 ng/mL to about 12 ng/mL of nicotine.

In some embodiments, the subject is a human who smokes more than 10 cigarettes a day. In some embodiments, the human smokes more than 20 cigarettes a day. In another embodiment, the human suffers from nicotine dependence. In yet another embodiment, the human is at an age of above 20. In one embodiment, the human suffers from tobacco withdrawal symptoms such as one or more of anxiety, irritability, restlessness, cravings, dizziness, impaired concentration, weight increase, emotional liability, somnolence and fatigue, increased sweating, and insomnia.

In some embodiments, the nicotine composition is applied to the skin. In some embodiments, the skin includes, but is not limited to, an upper arm, a shoulder, or an upper back.

In some embodiments, the nicotine composition is brushed on to the skin once or twice a day in an amount of from about 0.5 mL to about 5.0 mL. In some embodiments, the nicotine composition is brushed on to the skin one to four times a day for a period of time of 1 day to 365 days. In a preferred embodiment, the nicotine composition is brushed on to the skin once a day for about 84 days. In some embodiments, about 0.1 gram to 10 grams of the nicotine composition are brushed to the skin each time.

In some embodiments, the ratio of peak concentration and trough concentration of nicotine in the serum of the subject after application is less than about 10.

The present disclosure also provides a liquid composition comprising about 0.1% to about 20% by weight of nicotine, the composition, when applied to the skin, achieves one or more of the following: a) has a thickness of about 0.1 μm to about 10 μm in solid or semi-solid form; b) forms a solid or semi-solid film; and c) provides a mean Tₘₐₓ of nicotine from about 0.5 to about 24 hours. The liquid nicotine composition seeps into skin pores in liquid form and creates a biomechanical integration with the interior of said skin pores in solid form.

In another embodiment, the liquid nicotine composition further comprises pyroxilin, ether, and alcohol.

In some embodiments, the liquid nicotine composition provides a maximum serum nicotine concentration (Cₘₐₓ) following administration from about 5.0 ng/mL to about 50.0 ng/mL of nicotine, about 10 ng/mL to about 30 ng/mL of nicotine, or about 15 ng/mL to about 20 ng/mL of nicotine.

The present disclosure also provides a method for transpore delivery of insulin to a diabetic patient, the method comprising applying a liquid composition comprising about 0.1% to about 20% by weight of insulin to the skin of the subject, wherein the insulin composition, when administered to the subject, achieves one or more of the following: a) has a thickness of about 0.1 μm to about 10 μm in solid form; b) forms a solid or semi-solid film; and c) provides a mean Tₘₐₓ of insulin from about 1 hour to about 24 hours; wherein the liquid insulin composition seeps into skin pores in liquid form and creates a biomechanical integration with the interior of said skin pores in solid form.

In some embodiments, the area of skin application is about 1 cm² to about 500 cm ².

In some embodiments, administration results in at least 10% of the applied insulin entering the systemic circulation of the subject after about 10 to about 24 hours of contact on the skin.

In some embodiments, the film has a thickness of about 1 μm to about 5 μm. In one embodiment, the film is an occlusive film.

In some embodiments, the insulin composition provides a maximum serum insulin concentration (Cₘₐₓ) following administration from about 20.0 μU/mL to about 140.0 μU/mL of insulin. In some embodiments, the maximum serum insulin concentration (Cₘₐₓ) following administration is from about 30 μU/mL to about 100 μU/mL of insulin. In some embodiments, the maximum serum insulin concentration (Cₘₐₓ) following administration is from about 35 μU/mL to about 70 μU/mL of insulin. In some embodiments, the insulin composition provides a mean plasma concentration of insulin following administration from about 40 μU/mL to about 60 μU/mL of insulin.

In some embodiments, the subject is a human. In some embodiments, the human is an adult human. In some embodiments, the human is a patient with diabetes mellitus or a patient with metabolic syndrome. In another embodiment, the human suffers from type I diabetes. In one embodiment, the human suffers from type II diabetes. In another embodiment, the human is an obese patient. In one embodiment, the human is a child at least two years old.

In some embodiments, the insulin composition is applied to the skin. In some embodiments, the skin includes, but is not limited to, the abdomen, a thigh, or an upper arm.

In some embodiments, the insulin composition is brushed on to the skin once or twice a day in an amount of from about 0.5 mL to about 5.0 mL. In some embodiments, the nicotine composition is brushed on to the skin once, twice, three, or four times a day for a period of time from 1 day to 365 days. In a preferred embodiment, the insulin composition is brushed on to the skin once a day for about 90 days. In some embodiments, about 0.1 gram to about 10 grams of the insulin composition is brushed to the skin each time.

In some embodiments, the ratio of peak concentration and trough concentration of insulin in the serum of the subject after application is less than about 10.

The present disclosure also provides a liquid composition comprising about 0.1% to about 20% by weight of insulin. The liquid insulin composition, when administered to a subject, achieves one or more of the following: a) has a thickness of about 0.1 μm to about 10 μm in solid or semi-solid form; b) forms a solid or semi-solid film; and c) provides a mean Tₘₐₓ of insulin from about 0.50 hour to about 24 hours. The liquid insulin composition seeps into skin pores in liquid form and creates a biomechanical integration with the interior of said skin pores in solid form.

In some embodiments, the liquid insulin composition provides a serum insulin concentration (Cₘₐₓ) following administration from about 20 μU/mL to about 140 μU/mL of insulin, about 30 μU/mL to about 100 μU/mL of insulin, or about 35 μU/mL to about 71 μU/mL of insulin.

### BRIEF DESCRIPTION OF THE DRAWINGS

FIG. 1A provides a sectional view of the structure of skin pores.

FIG. 1B provides a sectional view of the transpore delivery of liquid drug compositions into the skin pores.

### DETAILED DESCRIPTION OF THE INVENTION

### Definition

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

### Definition of General Terms

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### Definition of Specific Terms

The term “pharmaceutically acceptable” as used herein generally refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug.

The term “transpore delivery” generally refers to the delivery of an agent in and through the skin pores into the skin and systemic circulation.

The term “Cₘₐₓ” as used herein generally refers to the maximum plasma concentration of a drug after it is administered to a subject.

The term “Cₘᵢₙ” as used herein generally refers to the lowest concentration reached by a drug before the next dose is administered.

The term “Tₘₐₓ” as used herein generally refers to the time required to reach the maximal plasma concentration (“Cₘₐₓ”) after administration of a drug.

The term “C_{avg}” as used herein generally refers to the mean plasma concentration of a drug achieved by transpore delivery.

The term “treat,” “treating,” or “treatment” as used herein generally refers to the administration of a composition to a subject for therapeutic purposes.

The term “administration” or “administering” as used herein refers to the act of a physician or other medical professional prescribing a pharmaceutical composition of the invention for a subject.

The term “mean” generally refers to an average value in a patient population. For example, a “mean Cₘₐₓ” refers to an average of the maximum plasma concentrations of a drug in a patient population.

The term “occlusive film” generally refers to a solid or semi-solid film that is an impermeable thin layer of material that covers the skin.

The term “serum concentration” generally refers to the amount of a drug or other compound in the circulation, both bound to proteins and unbound, the latter of which generally corresponds to the therapeutically active fraction.

The term “bioavailability” generally refers to the rate and extent to which the active ingredient is absorbed from a drug product and becomes available at the site of action.

“Bioequivalence” is a term in pharmacokinetics generally used to assess the expected in vivo biological equivalence of two proprietary preparations of a drug. Two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same.

The term “testosterone replacement therapy” generally refers to the transdermal administration to a subject of all or a portion of the testosterone that is normally produced by a healthy subject.

The term “estrogen replacement therapy” generally refers to the transdermal administration to a subject of all or a portion of the estrogen that is normally produced by a healthy subject.

The term “insulin” generally refers to a polypeptide possessing at least some of the biological activity of insulin (e.g., ability to affect the body through insulin's primary mechanism of action). For example, an insulin may be a polypeptide such as insulin having an A-chain polypeptide and a B-chain polypeptide coupled to the A-chain polypeptide by disulfide bonds. In various embodiments of the present invention, the insulin polypeptide preferably possesses a majority of the biological activity of insulin, more preferably possesses substantially all of the biological activity of insulin, and most preferably possesses all of the biological activity of insulin.

The unit “μU/mL” is a dose unit for insulin, in which 1 U means a biological equivalent of 34.7 μg pure crystalline human insulin.

The term “opioid” is interchangeable with the term “opioid agonist” or “opioid analgesic” and shall include the base of the opioid, mixed agonist-antagonists, partial agonists, pharmaceutically acceptable salts thereof, stereoisomers thereof, ethers and esters thereof, and mixtures thereof.

### Pharmaceutical Compositions

The present disclosure provides a liquid composition which comprises at least one film forming polymer. In some embodiments, the liquid composition comprises at least two film-forming polymers. Examples of film-forming polymers include, but are not limited to, cellulose nitrates, cellulose esters, cellulose ethers, cellulose esters-ethers, cellulose acylate, polyquaternium hyaluronic acid, or any combinations thereof. In one embodiment, the film-forming polymer is pyroxylin.

In certain embodiments, the total weight percentage of the one or more film forming polymers in the composition is from about 1% to about 10%, from about 3% to about 10%, from about 5% to about 10%, from about 7% to about 10%, from about 1% to about 8%, from about 3% to about 8%, from about 5% to about 8%, from about 7% to about 8%, from about 1% to about 6%, from about 3% to about 6%, from about 5% to about 6%, from about 1% to about 4%, from about 2% to about 4%, or from about 1% to about 2%. In some embodiments, the total weight percentage of the one or more film forming polymers is about 1%, about 2%, about 3%, about 4%, about 5%, about 6%, about 7%, about 8%, about 9%, or about 10%.

In some embodiments, the liquid drug composition further comprises nitrocellulose, ether and alcohol. In certain embodiments, the total weight percentage of nitrocellulose, ether and alcohol is from about 50% to about 99%, from about 60% to about 99%, from about 70% to about 99%, from about 80% to about 99%, from about 90% to about 99%, from about 50% to about 90%, from about 60% to about 90%, from about 70% to about 90%, from about 80% to about 90%, from about 50% to about 80%, from about 60% to about 80%, from about 70% to about 80%, from about 50% to about 70%, or from about 60% to about 70%.

In some embodiments, the liquid drug composition comprises pyroxylin, ether and alcohol. In certain embodiments, the total weight percentage of pyroxylin, ether and alcohol is from about 50% to about 99%, from about 60% to about 99%, from about 70% to about 99%, from about 80% to about 99%, from about 90% to about 99%, from about 50% to about 90%, from about 60% to about 90%, from about 70% to about 90%, from about 80% to about 90%, from about 50% to about 80%, from about 60% to about 80%, from about 70% to about 80%, from about 50% to about 70%, or from about 60% to about 70%.

In some embodiments, the composition comprises about 1% to about 10% of pyroxylin by weight. In some embodiments, the composition comprises about 1% , about 2%, about 3%, about 4%, about 5%, about 6%, about 7%, about 8%, about 9%, about 10% of pyroxylin by weight. In some embodiments, the composition comprises about 40% to about 75% of ether by weight. In some embodiments, the composition comprises about 40% to about 50%, about 40% to about 60%, about 50% to about 60%, about 50% to about 75%, or about 60% to about 75% of ether by weight. In some embodiments, the composition comprises about 20% to about 30% of alcohol. In some embodiments, the composition comprises about 20%, about 21%, about 22%, about 23%, about 24%, about 25%, about 26%, about 27%, about 28%, about 29%, or about 30% of alcohol by weight. Examples of ethers include, but are not limited to, diethyl ether and polyoxyetheylene lauryl ether. Examples of alcohol include, but are not limited to, ethanol and isopropanol. In some embodiments, the proportion of the weight of alcohol to the weight of ether is about 1:4, about 1:3.5, about 1:3, about 1:2.5, or about 1:2. In one embodiment, the liquid composition comprises 4 g nitrocellulose in 100 mL of a mixture of 25 mL alcohol and 75 mL ether.

In some embodiments, the liquid composition has a sufficient amount of ether and alcohol to dissolve all the ingredients and is a clear solution. In some embodiments, the ratio of ether and alcohol is from about 0.001 to about 1,000, about 0.01 to about 500, about 0.1 to about 100, about 0.1 to about 50, about 0.2 to about 40, about 0.3 to about 30, about 0.4 to about 20, about 0.5 to about 15, about 0.6 to about 10, about 0.7 to about 5, about 0.8 to about 3, about 0.9 to about 2, or about 1 to about 1.5.

In some embodiments, the weight percentage of pyroxylin in the liquid composition is from about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 9%, about 1% to about 8%, about 1% to about 7%, about 1% to about 6%, about 1% to about 5%, about 1% to about 4%, about 1% to about 3%, or about 1% to about 2%.

In yet other embodiments, the liquid composition comprises one or more plasticizers. In certain embodiments, the total weight percentage of the one or more plasticizers is from about 1% to about 20%, from about 5% to about 20%, from about 10% to about 20%, from about 15% to about 20%, from about 1% to about 16%, from about 5% to about 16%, from about 10% to about 16%, from about 1% to about 12%, from about 5% to about 12%, from about 8% to about 12%, from about 1% to about 8%, or from about 4% to about 8%. In some embodiments, the total weight percentage of the plasticizer is about 1%, about 2%, about 3%, about 4%, about %, about 6%, about 7%, about 8%, about 9%, about 10%, about 11%, about 12%, about 13%, about 14%, about 15%, about 16%, about 17%, about 18%, about 19%, or about 20%.

Examples of suitable plasticizers for the liquid drug compositions include, but are not limited to, polyethylene glycol, propylene glycol, polyesters (e.g. poly (lactic acid) and poly(lactide-co-glycolide)), polyesteramides, diesters/triesters of acids, diesters/triesters of alcohols, and combinations thereof.

In some embodiments, the liquid drug composition comprises a penetration enhancer. Penetration enhancers can interact with the lipid domain of the stratum corneum, disrupting these, and causing fluidization. Examples of penetration enhancers include, but are not limited to, dimethylsulphoxide, azone, pyrrolidones, fatty acids, fatty alcohols, peptides and trypsin. The total weight percentage of the penetration enhancers in the liquid composition is from about 0 to about 20%. In certain embodiments, the weight percentage of the penetration enhancer in the liquid composition is from about 0 to about 18%, from about 0 to about 16%, from about 0 to about 14%, from about 0 to about 12%, from about 0 to about 10%, from about 0 to about 8%, from about 0 to about 6%, from about 0 to about 4%, from about 0 to about 2%, from about 0 to about 1%. In some embodiments, the liquid composition does not include a penetration enhancer.

In some embodiments, the liquid composition comprises a surfactant. Examples of surfactants include, but are not limited to, alkylglucosides, alkylmaltosides, alkylthioglucosides, lauryl macrogolglycerides, fatty acids, lower alcohol fatty acid esters, polyoxyethylene alkylphenols, polyethylene glycol fatty acids esters, polypropylene glycol fatty acid esters, glycerol fatty acid esters, acetylated, glycerol fatty acid esters, polyethylene glycol glycerol fatty acid esters, polyglyceryl fatty acid esters, polyoxyethylene sorbitan fatty acid esters, polyoxyethylene glycerides, polyoxyethylene sterols, polyoxyethylene vegetable oils, polyoxyethylene hydrogenated vegetable oils, reaction mixtures of polyols and at least one member of the group consisting of fatty acids, vegetable oils, hydrogenated vegetable oils, and sterols, sugar esters, sugar ethers, sucroglycerides, fatty acid salts, bile salts, phospholipids, phosphoric acid esters, carboxylates, sulfates, sulfonates, or a combination thereof.

In some embodiments, the liquid composition comprises at least one pharmaceutically acceptable excipient. Suitable excipients that may be used in the liquid compositions discussed herein are known in the art and include, but are not limited to, polypeptides, synthetic polymers, liposomes, transfersomes, ethosomes, niosomes, solid lipid nanoparticles, penetration enhancers, solubilizers such as C₂ to C₈ straight and branched chain alcohols, diols and triols, moisturizers and humectants such as glycerine, amino acids and amino acid derivatives, polyaminoacids and derivatives, pyrrolidone carboxylic acids and its salts and derivatives, surfactants such as sodium laureth sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrollidone, polyvinyl alcohol and acrylic polymers, or combinations of any of the above.

In some embodiments, the liquid composition comprises about 0.1% to about 20% by weight of a pharmaceutically acceptable excipient. In some embodiments, the liquid composition comprises about 1% to about 20%, about 3% to about 20%, about 5% to about 20%, about 8% to about 20%, about 10% to about 20%, about 12% to about 20%, about 15% to about 20%, about 18% to about 20%, about 0.1% to about 15%, about 1% to about 15%, about 3% to about 15%, about 5% to about 15%, about 8% to about 15%, about 10% to about 15%, about 12% to about 15%, about 0.1% to about 12%, about 1% to about 12%, about 3% to about 12%, about 5% to about 12%, about 8% to about 12%, about 10% to about 12%, about 8% to about 10%, about 0.1% to about 8%, about 1% to about 8%, about 3% to about 8%, about 5% to about 8%, about 0.1% to about 3%, about 1% to about 3%, or about 0.1% to about 1% by weight of a pharmaceutically acceptable excipient.

In some embodiments, the liquid composition comprises at least one organic solvent. In some embodiments, the liquid composition comprises at least two organic solvents. In some embodiments, the liquid composition comprises two volatile solvents. In some embodiments, the volatile solvent is, but not limited to, alkane, alkene, ether, ester, alcohol, nitrile, or acetone. In some embodiments, a suitable organic solvent includes, but is not limited to, ethyl acetate, ether, ethyl alcohol, isopropyl alcohol, propylene glycol, hexane, heptane, toluene, and combinations thereof. In a preferred embodiment, the liquid composition comprises ethyl alcohol and ether.

In some embodiments, the thickness of the film after application ranges from about 0.1 μm to about 10 μm, from about 0.1 μm to about 5 μm, from about 0.1 μm to about 2 μm, from about 0.5 μm to about 10 μm, from about 0.5 μm to about 5 μm, from about 0.5 μm to about 2 μm, from about 1 μm to about 10 μm, from about 1 μm to about 5 μm, from about 1 μm to about 2 μm, about 3 μm to about 10 μm, from about 3 μm to about 5 μm, about 5 μm to about 10 μm, from about 7 μm to about 10 μm. In a preferred embodiment, the thickness of the film ranges from about 3 μm to about 4 μm.

In some embodiments, the film formed by the liquid composition is an occlusive film. Occlusion refers to an impermeable film. An occlusive film blocks diffusional water loss from the skin, thereby increasing hydration of the stratum corneum. Maintenance of the structural integrity of the stratum corneum is critical to the skin's barrier function. Increasing stratum corneum hydration reduces the skin's barrier efficiency. Therefore, an occlusive film enhances the penetration of a topically administered drug through skin pores.

In some embodiments, the liquid drug composition comprises one active agent. The active agent may include, but is not limited to, testosterone, estrogen, an opioid, nicotine, or insulin. In some embodiments, the liquid composition may further comprise a second active agent. The concentration of each active agent in the composition is described in the sections separately below.

In certain embodiments, the liquid composition comprises about 0.001% to about 20%, about 0.01% to about 20%, about 0.1% to about 15%, about 0.5% to about 10%, about 1% to about 10%, about 3% to about 10%, about 5% to about 10%, about 7% to about 10%, about 9% to about 10%, about 0.001% to about 8%, about 0.01% to about 8%, about 0.1% to about 8%, about 0.5% to about 8%, about 1% to about 8%, about 3% to about 8%, about 5% to about 8%, about 7% to about 8%, about 0.001% to about 6%, about 0.01% to about 6%, about 0.1% to about 6%, about 0.5% to about 6%, about 1% to about 6%, about 3% to about 6%, about 5% to about 6%, about 0.001% to about 4%, about 0.01% to about 4%, about 0.1% to about 4%, about 0.5% to about 4%, about 1% to about 4%, about 3% to about 4%, about 0.001% to about 2%, about 0.01% to about 2%, about 0.1% to about 2%, about 0.5% to about 2%, about 1% to about 2%, about 0.001% to about 1%, about 0.01% to about 1%, about 0.1% to about 1%, or about 0.5% to about 1% by weight of an active agent.

### Methods of Transpore Delivery

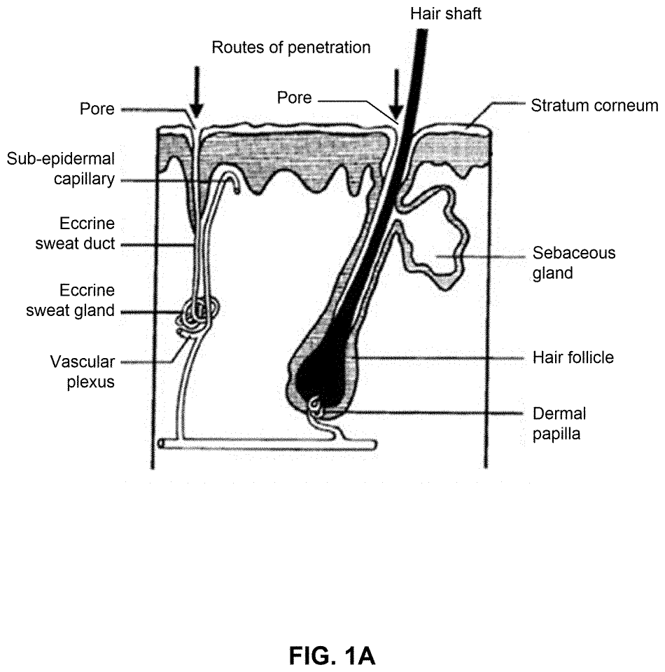

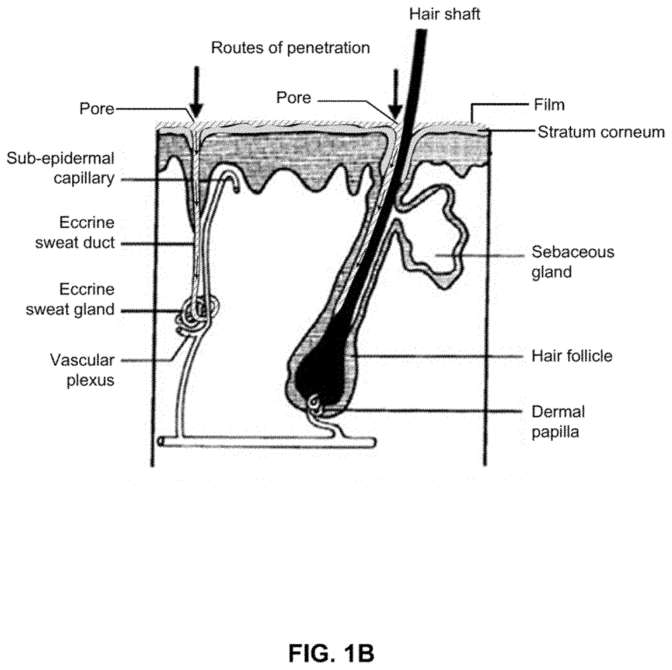

The skin is an important route for the delivery of drugs. Drug delivery through skin manages to avoid the variable absorption and metabolic breakdown associated with oral treatments and injections as the compound enters the systemic circulation without passing through the liver. Human skin is comprised of four main layers: the stratum corneum (SC); the viable epidermis; the overlying dermis; and the innermost subcutaneous tissues (hypodermis). FIG. 1 presents an illustration of normal human skin. The external layer of the skin (SC) functions as an effective barrier, and is essential for protection of the internal milieu from the external environment. Permeation through the SC is the rate limiting step in the dermal or transdermal delivery of drugs.

One route of transdermal delivery involves the movement of compounds into the skin through skin pores. Physiologically speaking, skin pores comprise the tiny ostia from either pilosebaceous follicles or eccrine sweat glands. A pilosebaceous follicle is a unit consisting of a hair follicle and a sebum-producing sebaceous gland. Eccrine sweat glands secrete water to the skin, where it cools the body by evaporation. There are two types of skin pores with different sizes: a pilosebaceous follicle has a diameter of approximately 40-80 μm, and an eccrine sweat gland has a diameter of approximately 5-10 μm. Hydrophilic and high molecular weight molecules, as well as particle-based drug delivery systems, can penetrate the skin through skin pores.

The present disclosure describes a method of systemic delivery of an active agent via skin pores. The method comprises applying a liquid composition that dries out to a solid or semi-solid film to the skin of a subject. When applied to the skin, the liquid composition seeps into the skin pores as well as covers the surface. The liquid dries as the solvent in the composition evaporates. The remaining polymer materials in the composition absorb local moisture, swell, and dry to a solid or semi-solid film. Thus, the composition creates a biomechanical integration with the microstructure of the skin. The film is tangible, yet barely visible, avoiding compliance issues and adheres in a peg-lock manner with the skin pores.

In one embodiment, the liquid composition once dried, can also be described as an intrapore drug-eluting stent or stent-like structure.

In some embodiments, the subject is preferably a mammal such as a non-primate, e.g., cow, pig, horse, cat, dog, rat, and a primate, e.g., a monkey such as a Cynomolgous monkey and a human.

Skin pores originate in the dermal tissue but are accessible on the surface of the skin. In essence, skin pores provide a passage way for an active agent to directly reach the dermis without having to traverse the intact barrier of the SC. In transpore delivery, the pharmaceutically active ingredient in the liquid composition travels through the skin pores to arrive at the viable epidermis and the dermis. The film that dries out from the liquid composition is sufficiently thin to contour the shape of each skin pore. This allows the film to sufficiently contact with the skin pores and enhances the efficiency of transpore delivery.

In some embodiments, upon application to skin, the liquid dries rapidly to form a clear, long-lasting, highly durable elastomeric film, adhering to the contours of the skin and providing a uniform film. Once brushed on the skin, the volatile components, diethyl ether and ethyl alcohol rapidly evaporate, leaving a thin transparent film on the skin. As the film adheres to the skin and dries, the drug/biologic-impregnated film permeates the pores of the skin, creating a transpore delivery system for the drug or biologic.

The effect of transpore delivery of a drug or biologic can be tested in a vasoconstrictor assay developed by McKenzie and Stoughton. See McKenzie A W, Stoughton R B. Method for comparing percutaneous absorption of steroids. Arch Dermatol 1962 86:608-10; also see Place V A, Velazquez J G, Burdick K H. Precise Evaluation of Topically Applied Corticosteroid Potency: Modification of the Stoughton-McKenzie Assay. Arch Dermatol. 1970;101(5):531-537.

In some embodiments, the liquid composition is applied to the skin by any common applicator such as a brush, roll, squeeze tube, sprayer or eye drop type of apparatus used to apply compositions to the skin. The compositions may also be applied by impregnating a porous base with the composition and wiping the resultant composition onto the skin area or where the porous base includes an adhesive, securing the porous base to the skin adjacent to the skin area, wherein the liquid composition is placed on the area to be treated.

In some embodiments, the composition used in the method of the present invention is a relatively low or high viscosity liquid which can be applied directly and accurately onto the skin area and does not require the application of additional pressure or rubbing as do certain creams and ointments that have been previously utilized. The term “viscosity” is the measure of fluid friction. A highly viscous material is one that possesses a great deal of internal friction, and will not pour or spread as easily as material of lesser viscosity. A typical range of suitable viscosities for the present liquid composition would be, for example, 0.1 to 5000 mPas, preferably 1 to 1000 mPas at 20° C.

In some embodiments, the liquid composition is applied to a skin area including, but not limited to, one or more of an axilla, shoulder, arm, neck, abdomen, buttock, chest, back, or thigh.

In some embodiments, the area of skin to which the composition is applied is from about 1 cm² to about 1000 cm², from about 1 cm² to about 500 cm², from about 1 cm² to about 300 cm², from about 1 cm² to about 200 cm², from about 1 cm² to about 100 cm², from about 1 cm² to about 50 cm², from about 1 cm² to about 25 cm², from about 1 cm² to about 10 cm², or from about 1 cm² to about 5 cm². In a preferred embodiment, the area of skin is from about 1 cm² to about 500 cm². In some embodiments, the composition must not be applied to face or groin areas.

One of the advantages of the film-forming composition is that once the liquid composition dries to an occlusive film, the film can remain on the skin for days to achieve a prolonged release of the active ingredient. Unlike traditional drug-release patches that are thick and not visually appealing, the film is so thin that it is barely noticeable as well as does not interfere with most daily activities of the patient. Because the film can stay on the skin for a prolonged time, it also eliminates the cumbersome need for repeated application by the patient.

In some embodiments, the occlusive film formed by the composition is kept on the skin for from 1 to 7 days, from 1 to 5 days, from 1 to 3 days, from 3 to 7 days, from 3 to 5 days, or from 5 to 7 days. The composition can be reapplied as needed if the film peels off the skin area. In one embodiment, the occlusive film is kept on the skin for 2-7 days.

In some embodiments, the composition is brushed on to the skin area multiple times daily including, but not limited to, once per day, twice per day, three times a day or four times a day. In some embodiments, the composition is brushed on to the skin in a single daily dose. In some embodiments, the composition is brushed on to the skin 1 to 7 times a week, 1 to 4 times a week, 1 to 2 times a week, 2 to 7 times a week, 2 to 4 times a week, 3 to 7 times a week, 3 to 5 times a week, 4 to 7 times a week, 4 to 5 times a week, or 5 to 7 times a week.

In some embodiments, the amount of the composition that is brushed on to the skin in a single dose is from about 0.05 to about 10 ml, from about 0.5 to about 5 ml, from about 0.5 to about 3 ml, from about 0.5 to about 1 ml, from about 0.5 to about 0.5 ml, from about 0.5 to about 10 ml, from about 0.5 to about 5 ml, from about 0.5 to about 3 ml, from about 0.5 to about 1 ml, from about 0.5 to about 1 ml, from about 1 to about 10 ml, from about 1 to about 5 ml, from about 1 to about 3 ml, from about 3 to about 10 ml, from about 3 to about 5 ml, from about 5 to about 10 ml, from about 5 to about 8 ml, or from about 7 to about 10 ml. In certain embodiments, the amount of liquid composition that is applied to the skin is a daily dose of about 0.05 ml, about 0.1 ml, about 0.5 ml, about 1 ml, about 2 ml, about 3 ml, about 4 ml, about 5 ml, about 6 ml, about 7 ml, about 8 ml, about 9 ml, or about 10 ml. In one embodiment, the amount of the composition that is brushed on to the skin is from about 0.5 to about 5 ml.

In certain embodiments, the amount of the active ingredient that is applied to the skin is a single dose from about 0.1 mg to about 10 mg, from about 0.1 mg to about 5 mg, from about 0.1 mg to about 3 mg, from about 0.1 mg to about 1 mg, from about 0.1 mg to about 0.5 mg, from 0.5 mg to about 10 mg, from about 0.5 to about 5 mg, from about 0.5 mg to about 3 mg, from about 0.5 mg to about 1 mg, from about 1 mg to about 10 mg, from about 1 mg to about 5 mg, from about 1 mg to about 3 mg, from about 3 mg to about 10 mg, from about 3 mg to about 7 mg, from about 3 mg to about 5 mg, from about 5 mg to about 10 mg, from about 5 mg to about 7 mg, from about 7 mg to about 10 mg, from about 0.05 mg to about 15 mg, from about 0.05 mg to about 10 mg, from about 0.05 mg to about 5 mg, from about 0.05 mg to about 1 mg, from about 0.05 mg to about 0.5 mg, from about 0.1 mg to about 20 mg, from about 0.1 mg to about 15 mg, from about 0.5 mg to about 20 mg, from about 0.5 mg to about 15 mg, from about 1 mg to about 20 mg, from about 1 mg to about 15 mg, from about 3 mg to about 20 mg, from about 3 mg to about 15 mg, from about 5 mg to about 20 mg, from about 5 mg to about 15 mg, from about 7 mg to about 20 mg, from about 7 mg to about 15 mg, from about 10 mg to about 20 mg, from about 10 mg to about 15 mg, from about 15 mg to about 20 mg, from about 5 mg to about 1000 mg, from about 5 mg to about 500 mg, from about 5 mg to about 100 mg, from about 5 mg to about 50 mg, from about 10 mg to about 1000 mg, from about 10 mg to about 500 mg, from about 10 mg to about 100 mg, from about 10 mg to about 50 mg, from about 50 to about 1000 mg, from about 50 to about 500 mg, from about 50 mg to about 100 mg, from about 100 mg to about 1000 mg, from about 100 mg to about 500 mg, or from about 500 mg to about 1000 mg.

In some embodiments, the liquid composition, when administered to a subject, results in about 0.5% to about 90% of the active ingredient entering the systemic circulation of the patient after about 8 to about10 hours of contact on the skin. In certain embodiments, the composition results in about 0.5% to about 80%, about 1% to about 70%, about 5% to about 60%, about 10% to about 50%, about 11% to about 45%, about 12% to about 40%, about 13% to about 35%, about 14% to about 30%, about 15% to about 25%, about 15% to about 22%, about 15% to about 20%, about 15% to about 18%, or about 15% to about 16% of the active ingredient entering the systemic circulation of the patient after 8 hours of contact on the skin. In certain embodiments, the liquid composition results in about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 19%, about 20%, about 21%, about 22%, about 23%, about 24%, or about 25% of the active ingredient entering the systemic circulation of the patient after 8 hours of contact on the skin.

Specific compositions vary and depend on the condition of the subject, symptom, disease, and active agent. Various embodiments of the invention are described in the sections below for each active agent.

### Pharmaceutical Testosterone Compositions

In some embodiments, the active agent in the liquid composition is testosterone. In some embodiments, the testosterone composition comprises about 0.5% to about 4%, about 1% to about 4%, about 1.5% to about 4%, about 2% to about 4%, about 2.5% to about 4%, about 3% to about 4%, about 3.5% to about 4%, about 0.5% to about 2%, or about 1% to about 2% by weight of testosterone.

When applied to the skin of a subject, the liquid composition delivers the testosterone into the epidermis, dermis, hypodermis or the systemic circulation of the subject via skin pores. In some embodiments, the liquid testosterone compositions can be formulated such that, when administered to a subject they provide a mean plasma concentration of testosterone ranging from about 300 ng/dL to about 1100 ng/dL. In yet another embodiment, the liquid testosterone compositions can be formulated such that, upon administration to a human male, provide a mean plasma concentration of testosterone ranging from about 350 ng/dL to about 800 ng/dL.

In some embodiments, the liquid testosterone compositions can be formulated such that, upon administration to a subject, provide a maximum serum testosterone concentration (Cₘₐₓ) of about 300 ng/dL to about 2500 ng/dL, about 300 ng/dL to about 2400 ng/dL, about 300 ng/dL to about 2300 ng/dL, about 300 ng/dL to about 2200 ng/dL, about 300 ng/dL to about 2100 ng/dL, about 300 ng/dL to about 2000 ng/dL, about 300 ng/dL to about 1900 ng/dL, about 300 ng/dL to about 1800 ng/dL, about 300 ng/dL to about 1700 ng/dL, about 300 ng/dL to about 1600 ng/dL, about 300 ng/dL to about 1500 ng/dL, about 300 ng/dL to about 1400 ng/dL, about 300 ng/dL to about 1300 ng/dL, about 300 ng/dL to about 1200 ng/dL, about 300 ng/dL to about 1100 ng/dL, about 300 ng/dL to about 1000 ng/dL, about 300 ng/dL to about 900 ng/dL, about 400 ng/dL to about 900 ng/dL, about 300 ng/dL to about 800 ng/dL, about 400 ng/dL to about 800 ng/dL, about 500 ng/dL to about 800 ng/dL, about 600 ng/dL to about 800 ng/dL, about 700 ng/dL to about 800 ng/dL, about 300 ng/dL to about 700 ng/dL, about 300 ng/dL to about 600 ng/dL, about 300 ng/dL to about 500 ng/dL, about 300 ng/dL to about 400 ng/dL, or about 300 ng/dL to about 350 ng/dL.

In some embodiments, the liquid testosterone compositions can be formulated such that, upon administration to a subject, the mean Tₘₐₓ is from about 0.5 to about 24 hours. In other embodiments, the liquid testosterone compositions can be formulated such that, upon administration to a subject, the Tₘₐₓ is from about 1 hour to about 24 hours, about 1 hour to about 23 hours, about 1 hour to about 22 hours, about 1 hour to about 21 hours, about 1 hour to about 20 hours, about 1 hour to about 19 hours, about 1 hour to about 18 hours, about 1 hour to about 17 hours, about 1 hour to about 16 hours, about 1 hour to about 15 hours, about 1 hour to about 14 hours, about 1 hour to about 13 hours, about 1 hour to about 12 hours, about 1 hour to about 11 hours, or about 1 hour to about 10 hours.

The liquid testosterone compositions of the invention can also include one or more excipients selected from binders, bufferants, diluents, disintegrants, colorants, resins, pH modifiers, lubricants, glidants, thickening agents, opacifying agents, humectants, desiccants, effervescing agents, plasticizing agents and the like.

In a preferred embodiment, the liquid testosterone compositions comprise pyroxilin, ether, and alcohol.

### Transpore Delivery of Testosterone

The methods and compositions of the present invention are useful for treating subjects, particularly human males, or even more particularly males who suffer from testosterone deficiency or hypogonadism.

The liquid testosterone compositions of the present disclosure can be used to treat any condition associated with testosterone deficiency, including, but not limited to, complete absence, of endogenous testosterone. Examples of conditions associated with testosterone deficiency that can be treated include, but are not limited to, congenital or acquired primary hypogonadism, hypogonadotropic hypogonadism, cryptorchidism, bilateral torsion, orchitis, vanishing testis syndrome, orchidectomy, Klinefelter's syndrome, post castration, eunuchoidism, hypopituitarism, endocrine impotence, infertility due to spermatogenic disorders, impotence, male sexual dysfunction (MSD) including conditions such as premature ejaculation, erectile dysfunction, decreased libido, and the like, micropenis and constitutional delay, penile enlargement, appetite stimulation, testosterone deficiency associated with chemotherapy, testosterone deficiency associated with toxic damage from alcohol, testosterone deficiency associated with toxic damage from heavy metals, osteoporosis associated with androgen deficiency, and combinations thereof.

Other conditions that can be treated with the liquid testosterone compositions disclosed herein include idiopathic gonadotropin, hypogonadism due to luteotropin-releasing hormone (LHRH) deficiency, or pituitary hypothalamic injury from tumors, trauma, or radiation. Typically, these subjects have low serum testosterone levels but have gonadotropins in the normal or low range. In one embodiment, the compositions may be used to stimulate puberty in carefully selected males with clearly delayed puberty not secondary to pathological disorder. In another embodiment, the liquid testosterone compositions may be used in female-to-male transgender therapy to stimulate and maintain male physical and sexual characteristics including body muscle mass, muscle tone, bone density, body mass index (BMI), enhanced energy, motivation and endurance, psychosexual activity etc. In some embodiments, the testosterone compositions may be useful in providing hormonal male contraception.

Additionally, the liquid testosterone compositions disclosed herein can also be used to improve the quality of life of subjects suffering from conditions such as decreased libido, diminishing memory, anemia due to marrow failure, renal failure, chronic respiratory or cardiac failure, steroid-dependent autoimmune disease, muscle wasting associated with various diseases such as AIDS, preventing attacks of hereditary angioedema or urticaria; andropause, and palliating terminal breast cancer. In some situations, certain biomarkers such as for example, increased SHBG levels, can be used to diagnose a subject who may be in need of testosterone therapy. These biomarkers can be associated with conditions/disease states such as anorexia nervosa, hyperthyroidism, hypogonadism, androgen insensitivity/deficiency, alcoholic hepatic cirrhosis, primary biliary cirrhosis, and the like.

In some embodiments, subjects that can be treated with the liquid testosterone compositions of the present disclosure are human males. In particular, in one embodiment, the human male is at least 14 years of age. In another embodiment, the human male is an adult of at least age 30. In one embodiment, the subject is a transgender (female to) male. In another embodiment, the subject can be an adult male of at least age 50. In yet a further embodiment, the subject can be an adult male of at least age 60.

In some embodiments, the present disclosure provides a method for transpore delivery of testosterone to a subject in need of testosterone or replacement therapy, comprising applying a liquid composition comprising about 0.1% to about 20% by weight of testosterone to the skin of the subject, the composition, when administered to the subject, achieves one or more of the following: a) has a thickness of about 0.1 μm to about 10 μm in solid form; b) forms a solid or semi-solid film; and c) provides a mean Tₘₐₓ of testosterone from about 8 hour to about 24 hours; wherein the liquid testosterone composition seeps into skin pores and creates a biomechanical integration with the interior of said skin pores in solid form.

In one embodiment, said area of skin application is about 1 cm² to about 500 cm².

In some embodiments, administration results in at least 10% of the applied testosterone entering the systemic circulation of the subject after about 10 to about 24 hours of contact on the skin.

In some embodiments, the liquid testosterone compositions disclosed herein can be used to treat testosterone deficiency in human males. In one embodiment, the human male being treated has an average baseline plasma testosterone concentration of about 400 ng/dL or less, about 350 ng/dL or less, about 300 ng/dL or less, about 250 ng/dL or less, or about 190 ng/dL or less.

In some embodiments, the methods disclosed herein provide a plasma concentration of testosterone within a target mean plasma testosterone concentration range for a male subject. The method comprises the step of applying to the male subject an initial regimen including a daily dose of a liquid testosterone composition. In some embodiments, the method of the present disclosure provides a target mean plasma testosterone concentration ranging from about 300 ng/dL to about 1100 ng/dL.

In one embodiment, the target mean plasma testosterone concentration range is about 300 ng/dL to 1100 ng/dL on or after about day 84 following the start of the initial regimen. In another embodiment, the target mean plasma testosterone concentration range is about 300 ng/dL to about 1100 ng/dL on or after day 120 following the start of the initial regimen. In yet a further embodiment, the mean target plasma testosterone concentration range is about 300 ng/dL to about 1100 ng/dL on or after day 180 following the start of the initial regimen.

The methods disclosed herein provide desirable pharmacokinetic parameters. In one embodiment, the method provides a mean plasma testosterone concentration in the range of 300 ng/dL to 1100 ng/dL in 75% or more of hypogonadal males after 84 days from the start of the initial regimen, based on a minimum group size of 24 hypogonadal males. In another embodiment, the method provides a maximum serum testosterone concentration Cₘₐₓ of 1500 ng/dL or less in less than or equal to 85% of hypogonadal males based on a minimum group size of 24 hypogonadal males. In yet a further embodiment, the method provides a maximum serum testosterone concentration Cₘₐₓ in the range of 1800 ng/dL to 2500 ng/dL in about 5% or less of hypogonadal males after 84 days from the start of the initial regimen based on a minimum group size of 24 hypogonadal males. In yet a further embodiment, the method provides a maximum serum testosterone concentration Cₘₐₓ of 2500 ng/dL in about 1% or less of hypogonadal males after 84 days from the start of the initial regimen based on a minimum group size of 24 hypogonadal males.

In one embodiment, the method provides a steady state ratio of serum testosterone Cₘₐₓ to Cₘᵢₙ of about 10.0 or less based on single subject administration. In some embodiments, the steady state ratio of serum testosterone Cₘₐₓ to Cₘᵢₙ is about 1 to about 9, about 1 to about 8, about 1 to about 7, about 1 to about 6, about 1 to about 5, about 1 to about 4, about 1 to about 3, or about 1 to about 2.

### Pharmaceutical Estrogen Compositions

In some embodiments, the active agent in the liquid composition is estrogen. In some embodiments, the estrogen composition comprises about 0.1% to about 20%, about 0.5% to about 20%, about 1.0% to about 20%, about 2% to about 18%, about 4% to about 16%, about 6% to about 14%, about 8% to about 12%, or about 10% to about 11% by weight of estrogen.

In some embodiments, when applied to the skin of a subject, the liquid composition delivers the estrogen into the epidermis, dermis, hypodermis or the systemic circulation of the subject via skin pores. In some embodiments, the liquid estrogen compositions disclosed herein can be formulated such that, when administered to a subject they provide an average serum estrogen ranging from about 20 pg/mL to about 350 pg/mL. In yet other embodiments, the liquid estrogen compositions can be formulated such that, upon single administration to a subject, they provide an average serum estrogen ranging from about 30 pg/mL to about 150 pg/mL.

In some embodiments, the liquid estrogen compositions can be formulated such that, upon administration to a subject, provide a maximum serum estrogen concentration Cₘₐₓ of about 20 pg/mL to about 350 pg/mL, about 20 pg/mL to about 350 pg/mL, about 20 pg/mL to about 320 pg/mL, about 20 pg/mL to about 290 pg/, about 20 pg/mL to about 260 pg/mL, about 20 pg/mL to about 230 pg/mL, about 20 pg/mL to about 200 pg/mL, about 20 pg/mL to about 180 pg/mL, about 20 pg/mL to about 160 pg/mL, about 30 pg/mL to about 150 pg/mL, about 40 pg/mL to about 130 pg/mL, about 50 pg/mL to about 150 pg/mL, about 60 pg/mL to about 140 pg/mL, about 70 pg/mL to about 130 pg/mL, about 80 pg/mL to about 120 pg/mL, or about 90 pg/mL to about 110 pg/mL.

In some embodiments, the liquid estrogen compositions can be formulated such that, upon administration to a subject, the mean Tₘₐₓ is from about 10 hours to about 24 hours, about 11 hours to about 22 hours, about 12 hours to about 20 hours, about 13 hours to about 18 hours, about 14 hours to about 17 hours, or about 15 hours to about 16 hours.

In addition to the ingredients disclosed in the section of pharmaceutical composition in general, the liquid estrogen compositions of the current disclosure can also include one or more of other additives selected from binders, bufferants, diluents, disintegrants, colorants, resins, pH modifiers, lubricants, glidants, thickening agent, opacifying agent, humectants, desiccants, effervescing agents, plasticizing agents and the like.

In a preferred embodiment, the liquid estrogen compositions comprise pyroxilin, ether, and alcohol.

### Transpore Delivery of Estrogen

The methods disclosed herein can be used to treat humans, particularly human females, or even more particularly females who suffer from insufficient endogenous levels of estrogen.

Deficient levels of estrogen can occur for a variety of reasons. For example, deficient levels of estrogen may be caused by e.g. natural menopause, peri-menopause, post-menopause, hypogonadism, castration or primary ovarian failure. Low levels of estrogen, irrespective of the cause, can lead to an overall decreased quality of life for women. Symptoms, diseases and conditions range from merely being inconvenient to life threatening.

The liquid estrogen compositions disclosed herein can be used to treat conditions associated with estrogen deficiency. Examples of conditions associated with estrogen deficiency include, but are not limited to, transient symptoms, such as vasomotor signs and psychological symptoms. Vasomotor symptoms comprise but are not limited to hot flashes, sweating attacks such as night sweats, and palpitations. The vasomotor symptoms may be “mild”, “moderate” or “severe” as defined by the FDA guidelines (Guidance for Industry: Estrogen and Estrogen/Progestin Drug Products to Treat Vasomotor Symptoms and Vulvar and Vaginal Atrophy Symptoms—Recommendations for Clinical Evaluation; U.S. Department of Health and Human Services; Food and Drug Administration; CDER; January 2003.) Psychological symptoms of estrogen deficiency comprise, but are not limited to, insomnia and other sleep conditions, poor memory, loss of confidence, mood changes, anxiety, loss of libido, difficulties in concentration, difficulty in making decisions, diminished energy and drive, irritability and crying spells.

Other conditions that can be treated by the liquid estrogen compositions disclosed herein include permanent effects of estrogen deficiency. Permanent effects comprise physical changes such as urogenital atrophy, atrophy of the breasts, cardiovascular disease, changes in hair distribution, thickness of hair, changes in skin condition and osteoporosis. Urogenital atrophy, and associated conditions such as vaginal dryness, increase in vaginal pH and subsequent changes in flora, or events which lead to such atrophy including decreases in vascularity, fragmentation of elastic fibers, fusion of collagen fibers, or decreases in cell volume, are symptoms treated or alleviated by the methods described herein.

Additionally, in some embodiments, the methods disclosed herein are useful for the prevention and management of osteoporosis, most notably post-menopausal osteoporosis. Furthermore, bone demineralization, reduction of bone mass and density, thinning and interruption of trabeculae, and/or consequent increase in bone fractures or bone deformations are particularly relevant. The prophylactic treatment of osteoporosis is also contemplated using the liquid estrogen compositions disclosed herein.

In some embodiments, the present disclosure provides a method for transpore delivery of estrogen to a subject in need of estrogen or replacement therapy, comprising applying a liquid composition comprising about 0.1% to about 20% by weight of estrogen to the skin of the subject, the composition, when administered to the subject, achieves one or more of the following: a) has a thickness of about 0.1 μm to about 10 μm in solid form; b) forms a solid or semi-solid film; and c) provides a mean Tₘₐₓ of estrogen from about 10 hour to about 24 hours, wherein the liquid estrogen composition seeps into skin pores and creates a biomechanical integration with the interior of said skin pores in solid form.

In one embodiment, said area of skin application is about 1 cm² to about 500 cm².

In some embodiments, administration results in at least 1.25% of the applied estrogen entering the systemic circulation of the subject after about 10 to about 24 hours of contact on the skin.

In one embodiment, the subject is a human female at least 14 years of age. In another embodiment, the human female is an adult of at least age 30. In a further embodiment, the subject is an adult female of at least age 50. In yet a further embodiment, the subject is an adult female of at least age 60. In another embodiment, the subject is a transgender (male to) female.

In another embodiment, the human female is a postmenopausal woman. In a preferred embodiment, the human female is a hysterectomized postmenopausal woman. Hysterectomy is the surgical removal of the uterus. A total hysterectomy is removal of the uterus and cervix. A partial hysterectomy is removal of the uterus leaving the stump of the cervix (also called supra-cervical). A hysterectomy can be accompanied by surgical removal of the ovaries (oophorectomy). Removal of the female gonads and the ovaries is female castration. Women who undergo a total hysterectomy with bilateral salpingo-oophorectomy (removal of both ovaries, i.e. castration) lose most of their hormone production, including many estrogens and progestins. A woman who is undergoing natural menopause has intact and functional female organs, while a woman who has been hysterectomized and castrated does not. Accordingly, in the present context, the term “hysterectomized woman” refers to a woman who has undergone a total or partly hysterectomy.

In some embodiments, the methods disclosed herein provide a serum concentration of estrogen within a target maximum serum estrogen concentration Cₘₐₓ range for a female subject. The method comprises the step of applying to the female subject an initial regimen including a daily dose of a liquid estrogen composition. In some embodiments, the method of the present disclosure provides a target maximum serum concentration Cₘₐₓ ranging from about 20 pg/mL to about 350 pg/mL of estrogen.

In one embodiment, the target maximum serum estrogen Cₘₐₓ range can be from about 20 pg/mL to about 350 pg/mL on or after day 84 following the start of the initial regimen. In another embodiment, the target maximum serum estrogen Cₘₐₓ range can be from about 20 pg/mL to about 350 pg/mL on or after day 120 following the start of the initial regimen. In yet a further embodiment, the mean target maximum serum estrogen Cₘₐₓ range can be from about 20 pg/mL to about 350 pg/mL on or after day 365 following the start of the initial regimen.

In one embodiment, the method provides a steady state ratio of serum estrogen Cₘₐₓ to Cₘᵢₙ of about 10.0 or less based on single subject administration. In some embodiments, the steady state ratio of serum estrogen Cₘₐₓ to Cₘᵢₙ is about 1 to about 9, about 1 to about 8, about 1 to about 7, about 1 to about 6, about 1 to about 5, about 1 to about 4, about 1 to about 3, or about 1 to about 2.

### Pharmaceutical Opioid Compositions

In some embodiments, the active agent in the liquid composition is an opioid. The term “opioid” refers to both opiates (i.e., natural alkaloids found in the resin of the opium poppy) and synthetic substances, and is typically defined as any psychoactive chemical that resembles morphine or other opiates in its pharmacological effects. Opioids function by binding to opioid receptors found principally in the central and peripheral nervous system and the gastrointestinal tract and the receptors in these organ systems mediate both the beneficial effects and the side effects of opioids. The analgesic (painkiller) effects of opioids are due to decreased perception of pain, decreased reaction to pain as well as increased pain tolerance.

Opioid compounds contemplated include, but are not limited to, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavretum, pentazocine, phenadoxone, phenomorphan, phenazocine, phenoperidine, piminodine, piritramide, propheptazine, promedol, properidine, propiram, propoxyphene, sufentanil, tramadol, tilidine, salts thereof, mixtures of any of the foregoing, mixed mu-agonists/antagonists, mu-antagonist combinations, and the like. The opioid may be in the form of the free base, a salt, a complex, etc. In certain preferred embodiments, the opioid is selected from the group consisting of hydromorphone, oxycodone, dihydrocodeine, codeine, dihydromorphine, morphine, buprenorphine, salts of any of the foregoing, and mixtures of any of the foregoing.

In some embodiments, the liquid opioid composition comprises about 0.1% to about 20%, about 0.5% to about 20%, about 1.0% to about 20%, about 2% to about 18%, about 4% to about 16%, about 6% to about 14%, about 8% to about 12%, or about 10% to about 11% by weight of opioid. In one embodiment, the opioid is buprenorphine.

In some embodiments, when applied to the skin surface of a subject, the liquid opioid composition delivers an opioid into the epidermis, dermis, hypodermis or systemic circulation of the subject via skin pores. In some embodiments, the liquid opioid compositions can be formulated such that, when administered to a subject provide a mean plasma concentration of opioid ranging about 50 pg/mL to about 1000 pg/mL of an opioid. In one embodiment, the opioid is buprenorphine. In yet other embodiments, the liquid buprenorphine compositions can be formulated such that, upon single administration to a human, provide a mean plasma concentration of buprenorphine ranging from about 50 pg/mL to about 1000 pg/mL.

In some embodiments, the liquid opioid compositions can be formulated such that, upon administration to a subject, provide a maximum serum opioid concentration Cₘₐₓ of about 100 pg/mL to about 1000 pg/mL, about 100 pg/mL to about 900 pg/mL, about 100 pg/mL to about 800 pg/mL, about 100 pg/mL to about 700 pg/mL, about 100 pg/mL to about 600 pg/mL, about 100 pg/mL to about 500 pg/mL, about 200 pg/mL to about 700 pg/mL, about 200 pg/mL to about 600 pg/mL, about 200 pg/mL to about 500 pg/mL, about 200 pg/mL to about 400 pg/mL, or about 300 pg/mL to about 400 pg/mL. In a preferred embodiment, the opioid is buprenorphine. In one embodiment, the liquid buprenorphine compositions can be formulated such that, upon administration to a subject, provide a mean plasma concentration of opioid ranging from about 50 pg/mL to about 1000 pg/mL.

In some embodiments, the liquid opioid compositions disclosed herein provide, upon administration to a subject, a mean Tₘₐₓ is from about 1 day to about 7 days. In other embodiments, the liquid opioid compositions can be formulated such that, upon administration to a subject, the time Tₘₐₓ is from about 2 days to about 6 days, about 3 days to about 5 days, or about 3 days to about 4 days.

In addition to the excipients disclosed in the section of pharmaceutical composition in general, the liquid opioid compositions of the current disclosure can also include one or more of other additives selected from binders, bufferants, diluents, disintegrants, colorants, resins, pH modifiers, lubricants, glidants, thickening agent, opacifying agent, humectants, desiccants, effervescing agents, plasticizing agents and the like.

In a preferred embodiment, the liquid opioid compositions comprise pyroxilin, ether, and alcohol.

### Transpore Delivery of Opioids

The methods and compositions disclosed herein are useful to treat a human, or even more particularly a patient who is in need of analgesic treatment, such as pain.

In some embodiments, the present disclosure provides a method of treating pain in a patient, comprising applying a liquid composition comprising about 0.1% to about 20% by weight of opioid to the skin of the subject, the composition, when administered to the subject, achieves one or more of the following: a) has a thickness of about 0.1 μm to about 10 μm in solid form; b) forms a solid or semi-solid film; and c) provides a mean Tₘₐₓ of opioid from about 1 day to about 7 days, wherein the liquid opioid composition seeps into skin pores and creates a biomechanical integration with the interior of said skin pores in solid form. In some embodiments, the contact period is from about 10 hours to about 24 hours, about 1 day to about 2 days, or about 2 days to about 3 days.

In one embodiment, said area of skin application is about 1 to about 500 cm².

In some embodiments, administration results in at least 10% of the applied opioid entering the systemic circulation of the subject after about 10 to about 24 hours of contact on the skin.

In some embodiments, the methods and liquid opioid compositions of the present disclosure may be used to treat acute or chronic pain, including neuropathic pain or nociceptive pain. Further, mixed pain states comprising neuropathic pain and nociceptive pain may be effectively treated.

In another embodiment, the present disclosure provides a method of treating pain in a patient whose pain is severe enough to require daily, around-the-clock, long-term opioid treatment and for which alternative treatment options are inadequate.

In some embodiments, the methods disclosed herein provide a serum concentration of opioid within a target maximum serum opioid concentration Cₘₐₓ range for a subject. The method comprises the step of applying to the subject an initial regimen including a daily dose of a liquid opioid composition. In some embodiments, the method of the present disclosure provides the target maximum serum concentration Cₘₐₓ of about 100 pg/mL to about 1000 pg/mL, about 200 pg/mL to about 700 pg/mL, or about 300 pg/mL to about 400 pg/mL. In one embodiment, the method of the present disclosure provides a mean plasma concentration of opioid following administration from about 50 pg/mL to about 1000 pg/mL.

In one embodiment, the liquid opioid composition is applied to the skin of a subject for a period of time from about 1 day to about 365 days. In a preferred embodiment, the period of time is about 84 days.

In one embodiment, the method provides a steady state ratio of serum opioid Cₘₐₓ to Cₘᵢₙ of about 10.0 or less based on single subject administration. In some embodiments, the steady state ratio of serum opioid Cₘₐₓ to Cₘᵢₙ is about 1 to about 9, about 1 to about 8, about 1 to about 7, about 1 to about 6, about 1 to about 5, about 1 to about 4, about 1 to about 3, or about 1 to about 2.

### Pharmaceutical Nicotine Compositions

In some embodiments, the active agent in the liquid composition is nicotine, nicotine free base or a nicotine salt. In some embodiments, the liquid nicotine composition comprises about 0.1% to about 20%, about 0.5% to about 20%, about 1.0% to about 20%, about 2% to about 18%, about 4% to about 16%, about 6% to about 14%, about 8% to about 12%, or about 10% to about 11% by weight of nicotine.

In some embodiments, when applied to the skin surface of a subject, the liquid composition delivers the nicotine into the epidermis, dermis, hypodermis or the systemic circulation of the subject via skin pores. In some embodiments, the liquid nicotine compositions can be formulated such that, when administered to a subject they provide a mean plasma concentration of nicotine ranging from about 2.5 ng/mL to about 12 ng/mL. In yet other embodiments, the liquid nicotine compositions can be formulated such that, upon single administration to a subject, provide a mean plasma concentration of nicotine ranging from about 5 ng/mL to about 10 ng/mL.

In some embodiments, the liquid nicotine compositions can be formulated such that, upon administration to a subject, provide a maximum serum nicotine concentration Cₘₐₓ of about 5.0 ng/mL to about 50 ng/mL, about 5.0 ng/mL to about 45 ng/mL, about 5.0 ng/mL to about 40 ng/mL, about 5.0 ng/mL to about 35 ng/mL, about 5.0 ng/mL to about 30 ng/mL, about 10 ng/mL to about 30 ng/mL, about 10 ng/mL to about 25 ng/mL, about 10 ng/mL to about 20 ng/mL, or about 15 ng/mL to about 20 ng/mL.

In some embodiments, the liquid nicotine compositions can be formulated such that, upon administration to a subject, the mean Tₘₐₓ is about 1 hour to about 24 hours. In other embodiments, the liquid nicotine compositions can be formulated such that, upon administration to a subject, the mean Tₘₐₓ is from about 1 hour to about 20 hours, about 1 hour to about 18 hours, about 1 hour to about 16 hours, about 1 hour to about 14 hours, about 1 hour to about 12 hours, about 2 hours to about 12 hours, about 3 hours to about 12 hours, about 4 hours to about 12 hours, about 5 hours to about 12 hours, about 6 hours to about 12 hours, about 7 hours to about 12 hours, about 8 hours to about 12 hours, about 9 hours to about 12 hours, about 10 hours to about 12 hours, or about 11 hours to about 12 hours.

In addition to the ingredients disclosed in the section of pharmaceutical composition in general, the liquid nicotine compositions of the current disclosure can further include one or more of other additives selected from binders, bufferants, diluents, disintegrants, colorants, resins, pH modifiers, lubricants, glidants, thickening agent, opacifying agent, humectants, desiccants, effervescing agents, plasticizing agents and the like.

In a preferred embodiment, the liquid nicotine compositions comprise pyroxilin, ether, alcohol.

### Transpore Delivery of Nicotine

In another aspect, the present disclosure provides a method for transpore delivery of nicotine for smoking cessation or nicotine cravings, comprising applying a liquid composition that dries to a solid or semi-solid film comprising about 0.1% to 20% by weight of nicotine to the skin of a human, the composition, when administered to the human, achieves one or more of the following: a) has a thickness of about 0.1 μm to about 10 μm in solid form; b) forms a solid or semi-solid film; and c) provides a mean Tₘₐₓ of nicotine from about 1 hour to about 24 hours, wherein the liquid opioid composition seeps into skin pores and creates a biomechanical integration with the interior of said skin pores in solid form. In some embodiment, the contact time on the skin is from about 10 mins to about 1 hour, about 2 hours to about 10 hours, about 10 hours to about 24 hours.

In one embodiment, said area of skin application is about 1 to about 500 cm².

In some embodiments, administration results in at least 10% of the applied nicotine entering the systemic circulation of the subject after about 10 to about 24 hours of contact on the skin.

In some embodiments, the methods and liquid nicotine compositions disclosed herein are useful to treat nicotine dependence. In another aspect, the present disclosure provides a method for replacing or substituting nicotine sources, such as cigarettes and chewing tobacco.

The method of the present disclosure are useful to treat a human, particularly humans 18 years of age or older, or adults, who smoke more than 10 cigarettes a day. In one embodiment, the method disclosed herein is useful to treat a human who smokes more than 20 cigarettes a day. In another embodiment, the method disclosed herein is useful to treat a human who suffers from nicotine dependence. In one embodiment, the method disclosed herein is useful to treat a human whose age is at least 21. In another embodiment, the human treated by the method disclosed here suffers from tobacco withdrawal symptoms selected from anxiety, irritability, restlessness, cravings, dizziness, impaired concentration, weight increase, emotional lability, somnolence and fatigue, increased sweating, insomnia, or combinations thereof.

In some embodiments, the method provides a serum concentration of nicotine within a target maximum serum nicotine concentration Cₘₐₓ range for a human. The method comprises the step of applying to the human an initial regimen including a daily dose of a liquid nicotine composition. In some embodiments, the method provides the target maximum serum concentration Cₘₐₓ of about 5.0 ng/mL to about 50.0 ng/mL, about 10 ng/mL to about 30 ng/mL, or about 15 ng/mL to about 20 ng/mL. In one embodiment, the method provides a mean plasma concentration of nicotine following administration from about 2.5 ng/mL to about 12 ng/mL.

In one embodiment, the present disclosure provides that the liquid nicotine composition may apply to the skin of a human for a period of time from about 1 day to about 365 days. In a preferred embodiment, the period of time is about 90 days.

In one embodiment, the method can provide a steady state ratio of serum nicotine Cₘₐₓ to Cₘᵢₙ of about 10.0 or less based on single subject administration. In some embodiments, the steady state ratio of serum nicotine Cₘₐₓ to Cₘᵢₙ is about 1 to about 9, about 1 to about 8, about 1 to about 7, about 1 to about 6, about 1 to about 5, about 1 to about 4, about 1 to about 3, about 1 to about 2.

### Pharmaceutical Insulin Compositions

In one aspect, the active agent in the liquid composition is insulin. In some embodiments, the insulin composition comprises about 0.1% to about 20%, about 0.5% to about 20%, about 1.0% to about 20%, about 2% to about 18%, about 4% to about 16%, about 6% to about 14%, about 8% to about 12%, or about 10% to about 11% by weight of insulin.

In some embodiments, when administered to the subject, the liquid composition delivers the insulin into the epidermis, dermis, hypodermis or the systemic circulation of the subject via skin pores. In some embodiments, the liquid insulin compositions of the present disclosure can be formulated such that, when administered to a subject they provide a mean plasma concentration of insulin ranging about 30μU/mL to about 100 μU/mL. In yet other embodiments, the liquid insulin compositions of the present disclosure can be formulated such that, upon single administration to a subject, they provide a mean plasma concentration of insulin ranging from about 40 μU/mL to about 60 μU/mL.

In some embodiments, the liquid insulin compositions of the present disclosure can be formulated such that, upon administration to a subject, they provide a maximum serum insulin concentration Cₘₐₓ ranging from about 20 μU/mL to about 140 μU/mL of insulin. In other embodiments, the liquid insulin compositions of the present disclosure can be formulated such that, upon administration to a subject, they provide a maximum serum insulin concentration Cₘₐₓ ranging from about 20 μU/mL to about 130 μU/mL of insulin, about 20 μ/mL to about 120 μU/mL of insulin, about 20 μU/mL to about 110 μU/mL of insulin, about 20 μU/mL to about 100 μU/mL of insulin, about 20 μU/mL to about 90 μU/mL of insulin, about 30 μU/mL to about 100 μU/mL of insulin, about 35 μU/mL to about 90 μU/mL of insulin, about 35 μU/mL to about 80 μU/mL of insulin, about 35 μU/mL to about 70 μU/mL of insulin, about 40 μU/mL to about 70 μU/mL of insulin, about 40 μU/mL to about 60 μU/mL of insulin, or about 40 μU/mL to about 50 μU/mL of insulin.

In some embodiments, the liquid insulin compositions of the present disclosure can be formulated such that, upon administration to a subject, the mean Tₘₐₓ is from about 1 hour to about 24 hours. In other embodiments, the liquid insulin compositions of the present disclosure can be formulated such that, upon administration to a subject, the mean Tₘₐₓ is from about 2 hours to about 22 hours, about 4 hours to about 20 hours, about 6 hours to about 18 hours, about 8 hours to about 16 hours, about 10 hours to about 14 hours, or about 12 hours to 13 hours.

In addition to the ingredients disclosed in the section of pharmaceutical composition in general, the liquid insulin compositions of the current disclosure can also include one or more of other additives selected from binders, bufferants, diluents, disintegrants, colorants, resins, pH modifiers, lubricants, glidants, thickening agent, opacifying agent, humectants, desiccants, effervescing agents, plasticizing agents and the like.

In a preferred embodiment, the liquid insulin compositions comprise pyroxilin, propyleneglycol, and alcohol.

### Transpore Delivery of Insulin

In one aspect, the methods and compositions are useful for treating diabetes mellitus. Diabetes mellitus is characterized by a broad array of physiologic and anatomic abnormalities, for example, abnormal insulin secretion, altered glucose disposition, altered metabolism of lipid, carbohydrates, and proteins, hypertension, neuropathy, retinopathy, abnormal platelet activity, and an increased risk of complications from vascular disease. Diabetics are generally divided into two categories. Patients who depend on insulin for the prevention of ketoacidosis have insulin-dependent diabetes mellitus (IDDM) or type 1 diabetes. Diabetics who do not depend on insulin to avoid ketoacidosis have non-insulin-dependent diabetes mellitus (NIDDM) or type 2 diabetes.

The diabetes and diabetes-related conditions which may be treated by the methods and liquid insulin formulations of the present disclosure include, but are not limited to, diabetes characterized by the presence of elevated blood glucose levels, for example, hyperglycemic disorders such as diabetes mellitus, including both type 1, type 2 and gestational diabetes as well as other hyperglycemic related disorders such as obesity, increased cholesterol, kidney related disorders, cardiovascular disorders and the like. Other forms of diabetes mellitus that may be treated and/or prevented using the methods and formulations of the invention include for example, maturity onset diabetes of youth, insulinopathies, diabetes associated with other endocrine diseases (such as Cushing's syndrome, acromegaly, glucagonoma, primary aldosteronism, insulin-resistant diabetes associated with acanthosis nigicans, lipoatrophic diabetes, diabetes induced by β-cell toxins, tropical diabetes, e.g., chronic pancreatitis associated with nutritional or toxic factors, diabetes secondary to pancreatic disease or surgery, diabetes associated with genetic syndrome, e.g., Prader-Willi Syndrome, diabetes secondary to endocrinopathies. Other diabetes-like conditions that may be treated using the methods of the invention include states of insulin resistance, with or without elevations in blood glucose, such as the metabolic syndrome that is associated with hypertension, lipid abnormalities and cardiovascular disease or polycystic ovarian syndrome.

In another aspect, the present disclosure relates to an improved transdermal administration method for delivering insulin to a subject, preferably humans, by directly targeting the skin, especially skin pores, whereby such method dramatically alters the pharmacokinetic (PK) and pharmacodynamic (PD) parameters of the administered insulin. Thus, the methods of the present disclosure are particularly useful for the treatment, prevention and/or management of diabetes mellitus such as insulin-dependent diabetes mellitus and/or non-insulin dependent diabetes mellitus. The methods of the present disclosure ameliorate one or more symptoms associated with diabetes mellitus.

In one aspect, the present disclosure provides a method for transpore delivery of insulin to a subject comprising applying a liquid composition that dries to a solid or semi-solid film comprising about 0.1% to about 20% by weight of insulin to the skin of the subject, the composition, when administered to the subject, achieves one or more of the following: a) has a thickness of about 0.1 μm to about 10 μm in solid form; b) forms a solid or semi-solid film; and c) provides a mean Tₘₐₓ of insulin from about 1 hour to about 24 hours, wherein the liquid insulin composition seeps into skin pores and creates a biomechanical integration with the interior of said skin pores in solid form. In some embodiments, the contact time of the film on skin is about 1 day to about 2 days, about 2 days to about 3 days, or about 3 days to about 4 days.

In one embodiment, said area of skin application is about 1 cm² to about 500 cm².

In some embodiments, administration results in at least 0.5% of the applied insulin entering the systemic circulation of the subject after about 10 to about 24 hours of contact on the skin.

Transpore delivery of insulin in accordance with the methods of the present disclosure provides an improved glycemic control and thus has an enhanced therapeutic efficacy in treatment, prevention and/or management of diabetes relative to traditional methods of insulin delivery, including subcutaneous insulin delivery. Preferably, the methods of the invention provide an improved glycemic control without an increase in hypoglycemic events. Although not intending to be bound by a particular mechanism of action, the improved glycemic control achieved using the transpore delivery methods of the invention is due, in part, to the control of both non-fasting (i.e., post prandial) and fasting glucose levels. The transpore delivery methods of the present disclosure lower fasting and/or post-prandial hyperglycemia more effectively than traditional methods of insulin delivery.

Directly targeting the skin, preferably skin pores, as taught by the present disclosure provides a much more easy administration of insulin. Another benefit of present disclosure is to achieve more rapid systemic distribution and offset of insulin.

The disclosure provides methods of treatment and/or prevention which involve administering a liquid insulin composition to a subject, preferably a mammal, and most preferably a human for treating, managing or ameliorating symptoms associated with diabetes mellitus. The methods disclosed here are useful for the treatment and/or prevention of diabetes or any related condition. In a preferred embodiment, the subject is a human. In another embodiment, the human treated by the method disclosed in this disclosure is an obese patient.

In one embodiment, the human is at least 2 years of age. In another embodiment, the human is an adult of at least age 20. In a further embodiment, the human an adult of at least age 50. In yet a further embodiment, the subject is an adult of at least age 60.

In some embodiments, the method provides a serum concentration of insulin within a target maximum serum insulin concentration Cₘₐₓ range for a subject. The method comprises the step of applying to the subject an initial regimen including a daily dose of a liquid insulin composition. In some embodiments, the method provides the target maximum serum concentration Cₘₐₓ of about 20 μU/mL to about 140 μU/mL, about 30 μU/mL to about 100 μU/mL, or about 35 μU/mL to about 70 μU/mL. In one embodiment, the method of the present disclosure provides the mean plasma concentration of insulin ranging from about 40 μU/mL to about 60 μU/mL.

In one embodiment, the target maximum serum insulin Cₘₐₓ range is about 20 μU/mL to about 140 μU/mL of insulin and is achieved by the method on or after day 84 following the start of the initial regimen. In another embodiment, the target maximum serum insulin Cₘₐₓ range can be about 20 μU/mL to about 140 μU/mL and is achieved by the method on or after about day 120 following the start of the initial regimen. In yet a further embodiment, the mean target maximum serum insulin Cₘₐₓ range can be about 20 μU/mL to about 140 μU/mL and can be achieved by the method on or after day 365 following the start of the initial regimen.

In one embodiment, the method can provide a steady state ratio of serum insulin Cₘₐₓ to Cₘᵢₙ of about 10.0 or less based on single subject administration. In some embodiments, the steady state ratio of serum insulin Cₘₐₓ to Cₘᵢₙ is about 1 to about 9, about 1 to about 8, about 1 to about 7, about 1 to about 6, about 1 to about 5, about 1 to about 4, about 1 to about 3, about 1 to about 2.

### EXAMPLES

The following examples are provided to promote a more clear understanding of certain embodiments of the present invention, and are in no way meant as a limitation thereon.

General procedure for transpore delivery of drugs: an active agent, eg. testosterone, estrogen, nicotine, epinephrine, etc, is dissolved in a nitrocellulose (collodion) solution. Upon application to skin, the liquid dries rapidly to form a clear, long-lasting, highly durable elastomeric film, adhering to the contours of the skin and providing a uniform film. Once brushed on the skin, the volatile components, such as diethyl ether and ethyl alcohol, rapidly evaporate, leaving a thin transparent film on the skin. As the film adheres to the skin and dries, the drug-impregnated film permeates the pores of the skin, creating a transpore delivery system for the drug

### Examples 1-4: Liquid Testosterone Compositions

a. Preparation of Liquid Testosterone Composition

Nitrocellulose (9 wt. %) is mixed with diethyl ether (20 wt. %). To this mixture is added anhydrous ethyl alcohol (70 wt. %), followed by testosterone (1 wt. %). The resultant solution is distributed into 20 mL clear vials and tightly closed with an appropriate lid. The vials are stored at 4° C.

b. Measurement of the Thickness

200 ul of the solution prepared above is taken out of the vial and mixed with 5 μl of 1% eosin Y and painted onto a coverslip. Images are collected using a Zeiss LSM 510 confocal microscope on samples that are in their liquid form and subsequently on samples that are allowed to dry. The dye is excited with HeNe 543 nm laser and Z-stack images are scanned under 560 nm long-pass filter with Zeiss Plan-Apochromat 63×/1.4 Oil immersion lens at intervals of 0.4 μm. Images are processed and measured with ImageJ.

c. Liquid Testosterone Compositions

Liquid testosterone compositions are prepared similarly to the method described in section a using the components set forth in Table I.

TABLE I

| Ingredients | Example 1 | Example 2 | Example 3 | Example 4 |
|---|---|---|---|---|
| Nitrocellulose | 10 wt. % | 15 wt. % | 5 wt. % | 5 wt. % |
| Testosterone | 1 wt. % | 1 wt. % | 1 wt. % | 2 wt. % |
| Ethyl Alcohol | 70 wt. % | 75 wt. % | 70 wt. % | 70 wt. % |
| Diethyl Ether | 19 wt. % | 19 wt. % | 24 wt. % | 23 wt. % |
| Total mL | 10 | 10 | 10 | 10 |

d. Clinical Test

The liquid testosterone compositions prepared in section c above are evaluated in a randomized multicenter, multi-dose, active and placebo controlled 90-day study in 50 adult males with morning testosterone levels ≤300 ng/dL. The study uses double-blinded for the doses of the liquid testosterone compositions and placebo, but open label for the non-scrotal testosterone transdermal system. During the first 60 days, patients are evenly randomized to placebo gel and the four liquid testosterone compositions. At Day 60, patients receiving testosterone are maintained at the same dose, or titrated up or down within their treatment group, based on 24-hour averaged serum testosterone concentration levels obtained on Day 30.

Of 32 hypogonadal men who are appropriately titrated with the liquid testosterone composition and who have sufficient data for analysis, about 70% achieve an average serum testosterone level within the normal range on treatment Day 90.

The mean testosterone concentrations on Day 30 for patients receiving testosterone and placebo will be expected in Table II.

TABLE II
Mean (±SD) Steady-State Serum
Testosterone Concentrations on Day 30

| | Example 1 | Example 2 | Example 3 | Example 4 | Placebo |
|---|---|---|---|---|---|
| | N = 10 | N = 10 | N = 10 | N = 10 | N = 10 |
| C_{avg} | 355 ± 50 | 360 ± 65 | 310 ± 75 | 610 ± 120 | 205 ± 42 |
| Cₘₐₓ | 540 ± 60 | 560 ± 75 | 480 ± 90 | 910 ± 150 | 260 ± 50 |
| Cₘᵢₙ | 220 ± 42 | 230 ± 55 | 210 ± 40 | 410 ± 55 | 160 ± 50 |

At Day 30, patients receiving Example 4 daily will show significant improvement from baseline in multiple sexual function parameters as measured by patient questionnaires when compared to placebo. These parameters include sexual motivation, sexual desire, sexual activity and spontaneous erections. For Example 4, improvements in sexual motivation, spontaneous erections, and sexual desire are maintained through Day 90. Sexual enjoyment and satisfaction with erection duration are improved compared to baseline but these improvements will not be significant compared to the placebo group.

### Examples 5-6: Liquid Estrogen Compositions

a. Preparation of the Liquid Estrogen Composition

The preparation of the Estrogen compositions is similar to the preparation of the liquid testosterone composition shown in Example 1.

b. Two Liquid Estrogen Compositions

Two liquid estrogen compositions are prepared similarly to the method described in section a using the components set forth in table III.

TABLE III

| | | |
|---|---|---|
| Ingredients | Example 5 | Example 6 |

| | | | | |
|---|---|---|---|---|
| Nitrocellulose | 5 | wt. % | 4 | wt. % |
| Estrogen | 0.15 | wt. % | 0.3 | wt. % |
| Ethyl Ether | 24.85 | wt. % | 24.70 | wt. % |
| Ethyl Alcohol | 70 | wt. % | 70 | wt. % |

| | | |
|---|---|---|
| Total mL | 1 | 1 |

c. Clinical Test

In a multiple-dose study, 24 postmenopausal women are treated for 14 days with Examples 5 and 6 applying to the forearm. Serum concentrations of estradiol are monitored daily. Pharmacokinetics parameters for estradiol from Example 5 and 6, as assessed on Day 14 of this study, are shown in Table IV.

TABLE IV
Estradiol Pharmacokinetic Parameters on Day 14

| | Example 5 | Example 6 |
|---|---|---|
| | 1.50 mg | 3 mg |
| PK Parameter | N = 12 | N = 12 |
| C_{avg} (pg/mL) | 20 | 31 |
| Cₘₐₓ (pg/mL) | 35 | 58 |
| Cₘᵢₙ (pg/mL) | 11 | 18 |

### Examples 7-8: Liquid Buprenorphine Compositions

a. Preparation of the Liquid Buprenorphine Composition

The preparation of the buprenorphine composition is similar to the preparation of the liquid testosterone composition showed in Example 1, section a.

b. Two Liquid Buprenorphine Compositions

Two liquid buprenorphine compositions are prepared similarly to the method described in section a using the components set forth in table V.

TABLE V

| Ingredients | Example 7 | Example 8 |
|---|---|---|
| Nitrocellulose | 5 wt. % | 5 wt. % |
| Buprenorphine | 1 wt. % | 2 wt. % |
| Ethyl Ether | 20 wt. % | 15 wt. % |
| Ethyl Alcohol | 70 wt. % | 70 wt. % |
| Total mL | 10 | 10 |

c. Clinic Test

The effect of Example 7 and 8 are evaluated in a single-dose study in 24 healthy male and female subjects aged 18 to 55 years.

Each example provides delivery of buprenorphine for 7 days. Pharmacokinetics parameters for buprenorphine from Example 7 and 8, as are assessed on Day 7 of this study, are summarized in Table VI.

TABLE VI
Pharmacokinetic Parameters of Buprenorphine in Healthy Subjects

| PK parameters | Example 7 | Example 8 |
|---|---|---|
| Cₘₐₓ (pg/mL) | 190 | 470 |
| Tₘₐₓ (d) | 3 | 3 |

### Examples 9-10: The Liquid Nicotine Compositions

a. Preparation of the Liquid Nicotine Compositions

The preparation of the nicotine compositions is similar to the preparation of the liquid testosterone composition showed in Example 1, section a.

b. Two Liquid Nicotine Compositions

Two liquid nicotine compositions will be prepared similarly to the method described in section a using the components set forth in table VII.

TABLE VII

| Ingredients | Example 9 | Example 10 |
|---|---|---|
| Nitrocellulose | 5 wt. % | 4 wt. % |
| Nicotine | 1 wt. % | 2 wt. % |
| Ethyl Ether | 24 wt. % | 24 wt. % |
| Ethyl Alcohol | 70 wt. % | 70 wt. % |
| Total mL | 10 | 10 |

c. Clinic Test

Smokers ranging in age from 20 to 60 years are treated with examples 9 and 10 for 24 hours. The pharmacokinetic parameters are shown in Table VIII.

TABLE VIII
Nicotine Pharmacokinetic Parameters

| PK Parameter | Example 9 | Example 10 |
|---|---|---|
| Tₘₐₓ (hours) | 6 | 12 |
| Cₘₐₓ (ng/mL) | 18 | 30 |

### Example 11: Liquid Insulin Compositions

a. Preparation of the Liquid Insulin Compositions

Nitrocellulose (10 wt. %) is mixed with ethyl alcohol (20 wt. %). To this solution is added propyleneglycol (65 wt. %), followed by insulin (5 wt. %). The resulted solution is distributed into multiple of 20 mL clear vials under Nitrogen gas. After distribution, the vials will be tightly closed with an appropriate lid. The vials are stored in refrigerator at −20° C.

b. Clinic Test

The liquid insulin composition showed in Example 11 will be administered topically in patients with type 2 diabetes and a body mass index (BMI) between 20 and 36 kg/m². The mean time to maximum concentration (Tₘₐₓ) is about 2 hours and the mean peak concentration (Cₘₐₓ) is about 84 μU/mL.

### Example 12 Vasoconstriction Test

The objective of the following studies is to evaluate transpore-delivery of various drugs.

### Example 12a Cortisone Vasoconstriction Test

Materials Used in the Cortisone Formulation are Listed Below:

| Ingredient | CAS Number | Percentage (%) |
|---|---|---|
| Nitrocellulose | CAS# 9004-70-0 | 23.76 |
| Diethyl Ether | CAS# 60-29-7 | 37.62 |
| Ethyl Alcohol | CAS# 64-1-5 | 37.62 |
| Cortisone | CAS# 53-06-5 | 1.00 |

Experimental Procedure: the biological effect of transpore-delivered cortisone was tested utilizing the FDA recommended method developed by McKenzie and Stoughton to assess the vasoconstriction effects of 1% cortisone. A Minolta Chroma Meter (CR-300) was used to measure the blanching of the skin. An area on the ventral area of the subject's forearm was selected due to the scarcity of hair and its relative uniform skin tone. Baseline lightness was measured in both test and control areas. Following the baseline measurements, the nitrocellulose film (without cortisone) was brushed on and allowed to dry on three (3) sites. The nitrocellulose formulation with cortisone was applied to three (3) other test sites. After one (1) hour, the film was removed and the blanching was measured. The mean percent change from baseline in skin blanching for the nitrocellulose film alone was −0.81±1.01 percent, and the mean percent change from baseline for the nitrocellulose with 1% cortisone was 1.38±0.85. Statistical analysis was performed using a two-tailed t-test. The results indicated that there was a significant difference between the nitrocellulose film alone and that containing the hormone cortisone (p=0.045).

Conclusion: these results demonstrate effective transpore-delivery of a biologically-active hormone.

### Example 12b Epinephrine Vasoconstriction Test

Materials Used in the Epinephrine Formulation are Listed Below:

| Ingredient | CAS Number | Percentage (%) |
|---|---|---|
| Nitrocellulose | CAS# 9004-70-0 | 23.99 |
| Diethyl Ether | CAS# 60-29-7 | 37.99 |
| Ethyl Alcohol | CAS# 64-1-5 | 37.99 |
| Epinephrine | CAS# 51-43-4 | 0.03 |

Experimental Procedure: The biological effect of transpore-delivered epinephrine was tested utilizing the FDA recommended method developed by McKenzie and Stoughton to assess the vasoconstriction effects of 0.025% epinephrine. A Minolta Chroma Meter (CR-300) was used to measure the blanching of the skin. An area on the ventral area of the subject's forearm was selected due to the scarcity of hair and its relative uniform skin tone. Baseline lightness was measured both test and control areas Following the baseline measurements, the nitrocellulose film (without epinephrine) was brushed on and allowed to dry on three (3) sites. The nitrocellulose formulation with epinephrine was applied to the three (3) other test sites. After one (1) hour, the film was removed and the blanching measured. The mean percent change from baseline in skin blanching for the nitrocellulose film alone was 2.02±0.51 percent, and the mean percent change from baseline for the nitrocellulose with 0.025% epinephrine was 8.32±2.54 percent. Statistical analysis was performed using a two-tailed t-test. The results indicated that there was a significant difference between the nitrocellulose film alone and that containing epinephrine (p=0.01).

Conclusion: these results demonstrate effective transpore-delivery of a vasoactive biological agent.

### Example 12c Nicotine Vasoconstriction Test

Materials Used in the Nicotine Formulation are Listed Below:

| Ingredient | CAS Number | Percentage (%) |
|---|---|---|
| Nitrocellulose | CAS# 9004-70-0 | 22.80 |
| Diethyl Ether | CAS# 60-29-7 | 36.10 |
| Ethyl Alcohol | CAS# 64-1-5 | 36.10 |
| Nicotine | CAS# 54-11-5 | 5.00 |

Experimental Procedure: The biological effect of transpore-delivered nicotine was tested utilizing the FDA recommended method developed by McKenzie and Stoughton to assess the vasoconstriction effects of 5% nicotine. A Minolta Chroma Meter (CR-300) was used to measure the blanching of the skin. An area on the ventral area of the subject's forearm was selected due to the scarcity of hair and its relative uniform skin tone. Baseline lightness was measured both test and control areas Following the baseline measurements, the nitrocellulose film (without nicotine) was brushed on and allowed to dry on three (3) sites. The nitrocellulose formulation with nicotine was applied to three (3) other test sites. After one (1) hour, the film was removed and the blanching measured. The mean percent change from baseline in skin blanching for the nitrocellulose film alone was −0.69±0.86 percent, and the mean percent change from baseline for the nitrocellulose with 5% nicotine was 2.21±2.42. Statistical analysis was performed using a two-tailed t-test. The results indicated that there was a significant difference between the nitrocellulose film alone and that containing the anti-addiction agent nicotine (p=0.05).

Conclusion: these results demonstrate effective transpore-delivery of an anti-addiction agent.

1-153. (canceled)

154. A liquid composition comprising about 0.5% to about 4% by weight of testosterone, wherein the composition, when administered to a subject, achieves one or more of the following:
a) forms a solid or semi-solid film;
b) has a thickness of about 0.1 μm to about 10 μm in solid or semi-solid form; and
c) provides a mean Tₘₐₓ of testosterone from about 1 hour to about 10 hours.

155. The composition of claim 154, further comprising pyroxilin, ether, and alcohol.

156. A method for transpore delivery of testosterone to a subject in need of replacement therapy comprising applying the liquid composition of claim 154 to the skin of the subject,
wherein said composition seeps into skin pores in liquid form and creates a biomechanical integration with the inside surface of said skin pores in solid form.

157. The method of claim 156, wherein the film has a thickness of about 1 μm to about 5 μm.

158. A liquid composition comprising about 0.1% to about 20% by weight of estrogen, wherein the composition, when administered to a subject, achieves one or more of the following:
a) forms a solid or semi-solid film;
b) has a thickness of about 0.1 μm to about 10 μm in solid or semi-solid form; and
c) provides a mean Tₘₐₓ of estrogen from about 10 hours to about 24 hours.

159. The composition of claim 158, further comprising pyroxilin, ether, and alcohol.

160. A method for transpore delivery of estrogen to a subject in need of replacement therapy comprising applying the liquid composition of claim 158 to the skin of a subject,
wherein said composition seeps into skin pores in liquid form and creates a biomechanical integration with the inside surface of said skin pores in solid form.

161. The method of claim 160, wherein the film has a thickness of about 1 μm to about 5 μm.

162. A liquid composition comprising about 0.1% to about 20% by weight of opioid, wherein the composition, when administered to a subject, achieves one or more of the following:
a) forms a solid or semi-solid film;
b) has a thickness of about 0.1 μm to about 10 μm in solid or semi-solid form; and
c) provides a mean T. of opioid from about 1 day to about 7 days.

163. The composition of claim 162, further comprising pyroxilin, ether, and alcohol.

164. A method for transpore delivery of an opioid to treat pain in a subject comprising applying the liquid composition of claim 162 to the skin of the subject,
wherein said composition seeps into skin pores in liquid form and creates a biomechanical integration with the inside surface of said skin pores in solid form.

165. The method of claim 164, wherein the film has a thickness of about 1 μm to about 5 μm.

166. A liquid composition comprising about 0.1% to about 20% by weight of nicotine, wherein the composition, when applied to a human, achieves one or more of the following:
a) forms a solid or semi-solid film;
b) has a thickness of about 0.1 μm to about 10 μm in solid or semi-solid form; and
c) provides a mean Tₘₐₓ of nicotine from about 0.50 hour to about 24 hours.

167. The composition of claim 166, further comprising pyroxilin, ether, and alcohol.

168. A method for transpore delivery of nicotine for smoking cessation comprising, applying the liquid composition of claim 166 to the skin of a human,
wherein said composition seeps into skin pores in liquid form and creates a biomechanical integration with the inside surface of said skin pores in solid form.

169. The method of claim 168, wherein said film has a thickness of about 1 μm to about 5 μm.

170. A liquid composition comprising about 0.1% to about 20% by weight of insulin, wherein the composition, when administered to a subject, achieves one or more of the following:
a) forms a solid or semi-solid film;
b) has a thickness of about 0.1 to about 10 μm in solid or semi-solid form; and
c) provides a mean Tₘₐₓ of insulin from about 0.50 hour to about 24 hours.

171. The composition of claim 170, further comprising pyroxilin, propyleneglycol, and alcohol.

172. A method for transpore delivery of insulin to a subject comprising applying the liquid composition of claim 170 to the skin of a subject,
wherein said composition seeps into skin pores in liquid form and creates a biomechanical integration with the inside surface of said skin pores in solid form.

173. The method of claim 172, wherein said film has a thickness of about 1 μm to about 5 μm.
